(19) United States
(12) Patent Application Publication
(10) Pub. No.: US 2023/0406883 A1
(43) Pub. Date: Dec. 21, 2023

(54) COMPOUNDS AND THEIR USE IN TREATMENT OF TACHYKININ RECEPTOR MEDIATED DISORDERS

(71) Applicant: Embark Biotech ApS, Copenhagen N (DK)

(72) Inventors: Magnus Bernt Fredrik Gustafsson, Frederiksberg (DK); Johnny Madsen, Copenhagen N (DK); Olivia Mulvad, Nordhavn (DK); Wouter Frederik Johan Hogendorf, Valby (DK); Jakob Bondo Hansen, Copenhagen V (DK)

(21) Appl. No.: 18/252,056

(22) PCT Filed: Nov. 9, 2021
(86) PCT No.: PCT/EP2021/081057

§ 371 Date: May 8, 2023

(30) Foreign Application Priority Data
Nov. 9, 2020 (EP) 20206509.0

(51) Int. Cl. C07K 7/22; A61K 47/54; A61P 3/10

(52) U.S. Cl. CPC C07K 7/22; A61K 47/542; A61P 3/10; A61K 38/00

(57) ABSTRACT

The present invention relates to compounds and their use in treatment of disorders mediated by tachykinin receptors, such as the tachykinin receptor 2.

### TECHNICAL FIELD

The present invention relates to compounds and their use in treatment of disorders mediated by tachykinin receptors, such as the tachykinin receptor 2.

### BACKGROUND

The family of Tachykinin neuropeptide receptors consists of three G protein-coupled receptors (GPCRs): Tachykinin receptor (Tacr) 1, Tacr2 and Tacr3, also known as Neurokinin receptor 1-3, (NK1-3R). The endogenous ligands for tachykinin receptors are the neuropeptides Substance P (SP) being the preferred ligand for NK1R, Neurokinin A (NKA) the preferred ligand for NK2R, and Neurokinin B the preferred ligand for NK3R. Neither of the endogenous ligands are specific for their receptor. Each peptide ligand can, thus, cross-activate all members of the Tachykinin receptor family with a potency close to the potency of its preferred receptor. Upon activation, the Tachykinin receptors preferentially couple to Gq, generating an intracellular inositol trisphosphate (IP₃) signaling response. All receptors can, in addition, also couple to Gs and induce cAMP accumulation, although with lower potency than Gq-activation.

Obesity, insulin resistance and type 2 diabetes are multifactorial diseases. The diseases are all interconnected, and the exact pathological mechanisms are unknown.

Obesity is widely accepted to be caused by an imbalance between energy intake and energy expenditure (EE). Thus, increased high caloric intake accompanied by inactivity is believed to be the main driver of obesity. In addition to the energy imbalance, high circulating insulin levels, as seen in insulin resistance, is believed to augment weight gain due to increased insulin-mediated nutrient storage.

Insulin resistance is a condition where cells of the body do not respond properly to the endocrine hormone insulin. The role of insulin is to allow the cells of the body to take up glucose to be used as energy fuel or for storage as fat. This means that when facing insulin resistance, the body is more likely to build up glucose in the blood leading to elevated blood glucose (hyperglycemia). As a result, the body produces more insulin trying to cope with the hyperglycemia, and therefore individuals with insulin resistance often produces more insulin compared to healthy individuals.

Diabetes is a disease where the body's insulin producing cells fail to meet the demand for insulin to regulate blood glucose. In general, diabetes can by stratified into three different types: Gestational diabetes, which is diabetes occurring during pregnancy. Type 1 diabetes, which is an autoimmune disorder where the beta-cells are destroyed and the individual is not able to produce insulin. Type 2 diabetes, the most common form of diabetes and caused by progressive beta-cell loss and insulin resistance. Beta-cell loss in type 2 diabetes is believed to be caused by beta-cell exhaustion, due to increased insulin demand, in combination cellular damage as a result of elevated blood glucose and circulating fatty acids.

Due to the interconnectivity of obesity, insulin resistance and diabetes, treatment that targets energy expenditure, i.e. by activating B/BAT, will have the potential to treat all three. Thus, increased energy expenditure has potential to treat obesity by decreasing energy stores, insulin resistance through decrease of fasting glucose, and diabetes by reducing fasting glucose per se and protect the beta-cells from exhaustion via reduced insulin demand.

Brown and beige adipose tissue (B/BAT) can be physiologically stimulated by cold exposure to significantly consume glucose and triglyceride-derived fatty acids from the blood and increase energy expenditure. Classically, brown and beige adipose tissue is activated upon stimulation of the Gs-coupled beta-adrenergic GPCRs, to elicit an intracellular cAMP response that activates lipolysis, glucose and lipid uptake from the periphery, and uncoupling of electron transport chain in the mitochondria by activating uncoupling protein 1. The uptake of lipids and glucose by activated brown and beige adipose tissue is superior to any other tissues, and activation of those tissues is therefore attractive for development of therapies for obesity, insulin resistance and diabetes.

Current attempts to activate B/BAT in humans have focused on the beta-adrenergic/cAMP pathway. This pathway has indeed proven capable of inducing substantial EE but with concomitant increases in unwanted side-effects such as heart rate, blood pressure and blood glucose (hyperglycemia).

In addition to B/BAT activation, the NK2R and ligand NKA is able to activate NK2Rs on visceral smooth muscle and stimulate contraction of colon and urinary bladder. The contractile activity of NK2R activation is conserved across species including rats, dogs, pigs, and humans.

Several studies in mouse, rat, dog and macaques have shown that NK2R agonists are gastrointestinal and bladder prokinetic agents causing dose-dependent smooth muscle contractions by activating NK2Rs located on smooth muscle cells. Emesis and hypotension are common side effects caused by NK1R cross activation and hence, development of NK2R specific agonists is desired to decrease side-effects in these therapies.

### SUMMARY

The present inventors have developed a series of compounds targeting the tachykinin/neurokinin receptor 2 (NK2R). NK2R is a member of the tachykinin G-protein coupled receptor (GPCR) family also containing tachykinin/neurokinin receptor 1 and 3 (NK1R and NK3R). The endogenous ligand for NK2R is neurokinin A (NKA), whereas substance P and neurokinin B are the endogenous ligands for NK1R and NK3R, respectively. NKA is a 10 amino acid, locally acting neuropeptide mainly produced in enterochromaffin cells and it is known to activate smooth muscle contraction. NK2R preferentially couples to Gq-proteins but can also recruit Gs and Gbeta-gamma and beta-arrestins. The primary organs of Tacr2 mRNA expression are adrenal glands (mice) and gastrointestinal tract (humans and mice).

The present inventors provide the synthesis of chemically stable agonists of NK2R as activators of energy expenditure for treatment of NK2R mediated disorders, such as a NK2R mediated disorder selected from the group consisting of: obesity, dysfunctional voiding, diabetes, such as type-II diabetes, and diabetes-related disorders.

In a first aspect, a compound according to formula (I) is provided:

(A)-(B) (I),

wherein;
(A) is a peptide comprising an amino acid sequence of the general formula X₁X₂X₃X₄X₅X₆X₇, wherein
X₁ is selected from the group consisting of: aspartic acid (D) and glutamic acid (E);
X₂ is selected from the group consisting of: lysine (K), arginine (R), and histidine (H);
X₃ is selected from the group consisting of: tyrosine (Y), phenylalanine (F), meta-tyrosine (m-Y), valine (V), tryptophan (W), methionine (M), leucine (L), isoleucine (I), and alanine (A);
X₄ is selected from the group consisting of: valine (V), threonine (T), serine (S), asparagine (N), glutamine (Q), glycine (G), and alanine (A);
X₅ is selected from the group consisting of: glycine (G), 2-aminoisobutyric acid (Aib), serine (S), alanine (A), valine (V), leuicine (L), beta-alanine (bA) and isoleucine (I);
X₆ is selected from the group consisting of: leucine (L), isoleucine (I), alanine (A) and N-methyl leucine (Me-Leu); and
X₇ is selected from the group consisting of: norleucine (Nle), methoxinine (Mox), methionine (M), 4-fluorophenylalanine (4fF), and 4-methoxyphenylalanine (4MeOF);

(B) is a conjugated moiety of the general formula (II)

Fa-Lg (II),
wherein;
Fa is a C₁₀-C₂₀ fatty acid, optionally substituted with one or more carboxylic acid groups,
Lg is a linking group, which covalently links (B) to the peptide (A),
and wherein (B) is covalently linked to a terminal amino acid or to a non-terminal amino acid.

In a second aspect, a pharmaceutical composition is provided comprising the compound as defined herein, and one or more pharmaceutically acceptable adjuvants, excipients, carriers, buffers and/or diluents.

In a third aspect, a compound is provided as defined herein for use as a medicament.

In a fourth aspect, a method for treating a disease in a subject is provided, comprising administering a compound as herein for treatment of a NK2R mediated disorder.

In a fifth aspect, a method for modulating the activity of NK2R is provided, comprising contacting NK2R with a compound as defined herein.

In a sixth aspect, a use of a compound as defined herein is provided for the manufacture of a medicament for the treatment of a metabolic disorder.

### DESCRIPTION OF DRAWINGS

FIG. 1: Position 6 (Xa): Phe-Tyr mutation. Substitution to tyrosine in NKA(4-10) analogues promotes hNK2R selectivity. Data from position 6 (Xa) mutations. Receptor activation was measured by IP₃-assay for compounds 304 and 305 on human (h)NK1R (FIG. 1, A), hNK2R (FIG. 1, B) or hNK3R (FIG. 1, C) and subjected to IP₃-assay using the indicated peptide compounds as agonists (ligands). Neurokinin A (NKA) was used with all receptors as a comparison, whereas Substance P (SP) and Neurokinin B (NKB) were used only with hNK1R and hNK3R, respectively. Graphs show receptor activation (³H-myoinositol signal) of indicated receptors after peptide compound incubation as a function of compound concentration (log[ligand]). Data are presented as mean ³H-myoinositol signal +/− SD. Nonlinear regression was performed with the Sigmoidal, 4PL, X is log(concentration) equation in Graphpad Prism 8.

FIG. 2: Position 7 (X₄) Val-Thr mutation. Threonine substitution on position 7 works as a selectivity driver independent of Tyr6 in NKA(4-10) analogues. Data from position 7 (X₄) mutations. Receptor activation was measured by IP3-assay for compounds 344, 366, 381, 382, 383 and 384 human (h)NK1R (FIG. 2, A), hNK2R (FIG. 2, B) or hNK3R (FIG. 2, C) and subjected to IP₃-assay using the indicated peptide compounds as agonists (ligands). Neurokinin A (NKA) was used with all receptors as a comparison, whereas Substance P (SP) and Neurokinin B (NKB) were used only with hNK1R and hNK3R, respectively. Receptor activation (³H-myoinositol signal as percent of 10⁻⁶ M NKA) of indicated receptors after peptide compound incubation as a function of compound concentration (log[ligand]). Data are presented as mean receptor activation (percent) +/− SD. Nonlinear regression was performed with the Sigmoidal, 4PL, X is log(concentration) equation in Graphpad Prism 8.

FIG. 3: Position 10 (X₇) mutation. Met substitution. Methionine substitution with norleucine or metoxinine improves hNK2R selectivity independent of selectivity-driver but slightly reduces hNK2R efficacy. Data from position 10 (X₇) mutations. Receptor activation was measured by IP3-assay for compounds 395, 316, 305, 344 and 394 on human (h)NK1R (FIG. 3, A), hNK2R (FIG. 3, B) or hNK3R (FIG. 3, C) and subjected to IP₃-assay using the indicated peptide compounds as agonists (ligands). Neurokinin A (NKA) was used with all receptors as a comparison, whereas Substance P (SP) and Neurokinin B (NKB) were used only with hNK1R and hNK3R, respectively. Receptor activation (³H-myoinositol signal as percent of 10⁻⁶ M NKA) of indicated receptors after peptide compound incubation as a function of compound concentration (log[ligand]). Data are presented as mean receptor activation (percent)+/−SD. Nonlinear regression was performed with the Sigmoidal, 4PL, X is log(concentration) equation in Graphpad Prism 8.

FIG. 4: Peptide analogues with neutral and positively charged linkers are preferred. Data from protractor linker charge analysis. Receptor activation was measured by IP₃-assay for compounds 305, 318, 319 and 321 on human (h)NK1R (FIG. 4, A), hNK2R (FIG. 4, B) or hNK3R (FIG. 4, C) and subjected to IP₃-assay using the indicated peptide compounds as agonists (ligands). Neurokinin A (NKA) was used with all receptors as a comparison, whereas Substance P (SP) and Neurokinin B (NKB) were used only with hNK1R and hNK3R, respectively. Receptor activation (³H-myoinositol signal as percent of 10⁴M NKA) of indicated receptors after peptide compound incubation as a function of compound concentration (log[ligand]). Data are presented as mean receptor activation (percent)+/−SD. Nonlinear regression was performed with the Sigmoidal, 4PL, X is log(concentration) equation in Graphpad Prism 8.

FIG. 5: Composition of protractor is important for receptor selectivity and in vivo half-life of N-terminal protracted NKA(4-10) analogues. Data from mono- or di-fatty acid analysis. Receptor activation was measured by IP₃-assay for compounds 305, 344, 390 and 391 on human (h)NK1R (FIG. 5, A), hNK2R (FIG. 5, B) or hNK3R (FIG. 5, C) and subjected to IP₃-assay using the indicated peptide compounds as agonists (ligands). Neurokinin A (NKA) was used with all receptors as a comparison, whereas Substance P (SP) and Neurokinin B (NKB) were used only with hNK1R and hNK3R, respectively. Receptor activation (³H-myoinositol signal as percent of 10⁴M NKA) of indicated receptors after peptide compound incubation as a function of compound concentration (log[ligand]). Data are presented as mean receptor activation (percent) +/− SD. Nonlinear regression was performed with the Sigmoidal, 4PL, X is log(concentration) equation in Graphpad Prism 8.

FIG. 6: NK2R agonism improves fasting blood glucose as well as glucose and insulin tolerance in die-induced obese mice. Wild type diet induced obese C57BL/6NRj mice were treated once with a subcutaneous injection of 344 (325 nmol/kg) and subjected to an intraperitoneal glucose tolerance test (ipGTT; FIG. 6, A.) or intraperitoneal insulin tolerance test (ipITT; FIG. 6, B.) 24 hours after treatment. Data are presented as means +/− SEM, n=5-7, analyzed by two-way ANOVA with Bonferroni's post-hoc test, *p<0.05, **p<0.01, **p<0.0001.

FIG. 7: NK2R corrects dysfunctional voiding in mice. Dysfunctional voiding was induced by oral gavage of Loperamide (LP; 5 mg/kg) 30 min prior to subcutaneous administration of different doses of selective NK2R agonist, compound 344. Vehicle (Veh) treated mice were used as control for normal voiding. Voiding was assessed as number of feces pellets produced during six hours. Data are presented as means +/− SEM, n=5-7, analyzed by one-way ANOVA with multiple comparison test of compound 344 treated versus LP, *p<0.01.

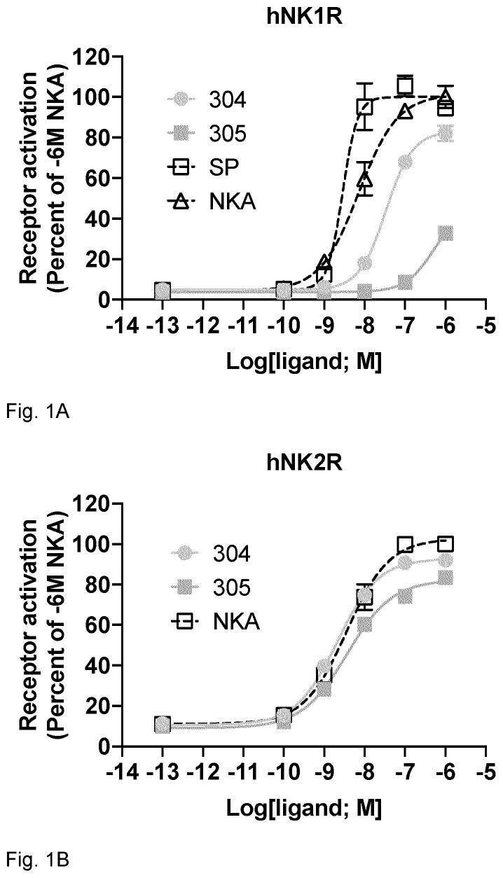

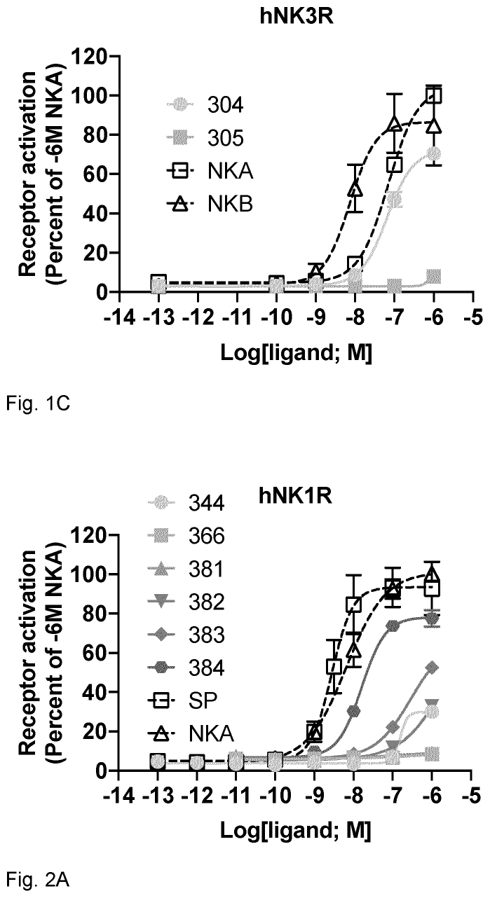

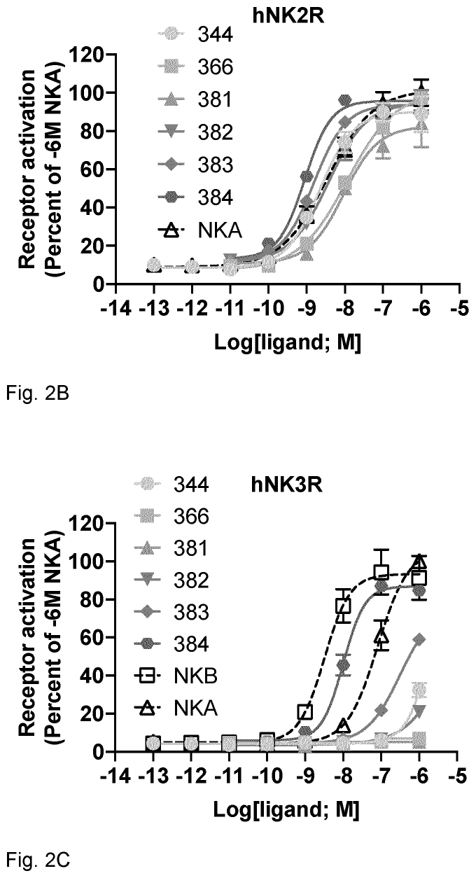

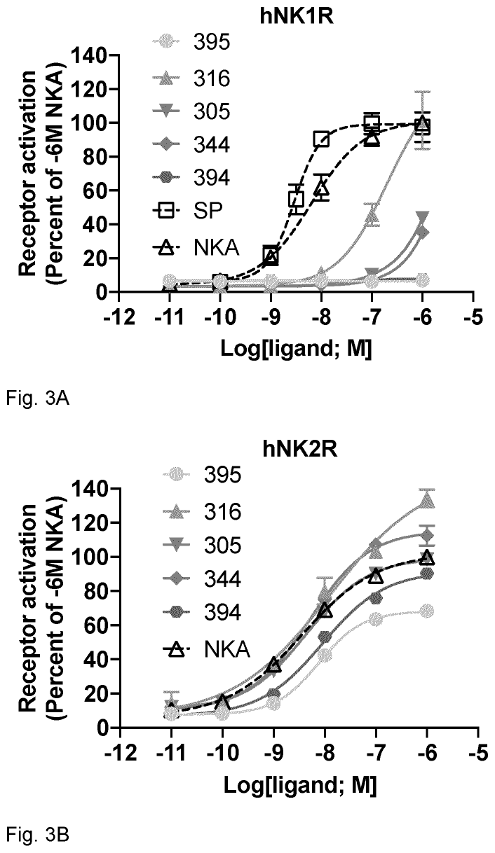

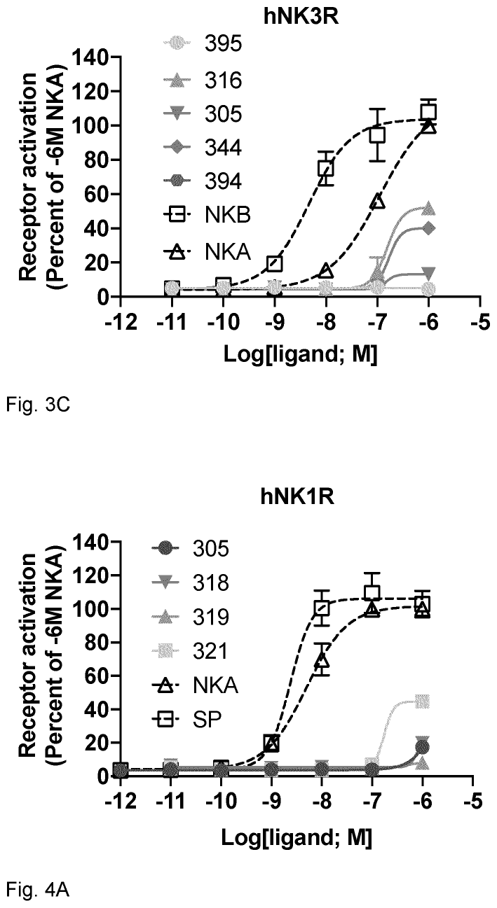

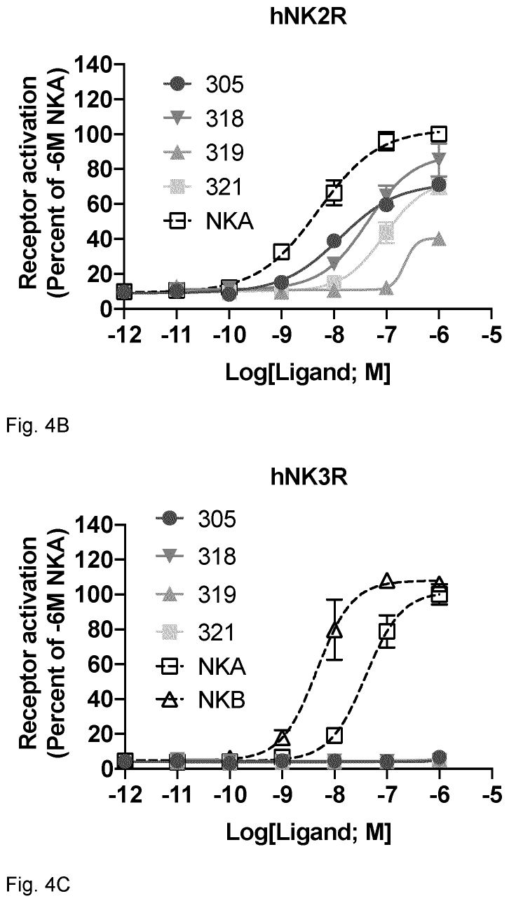

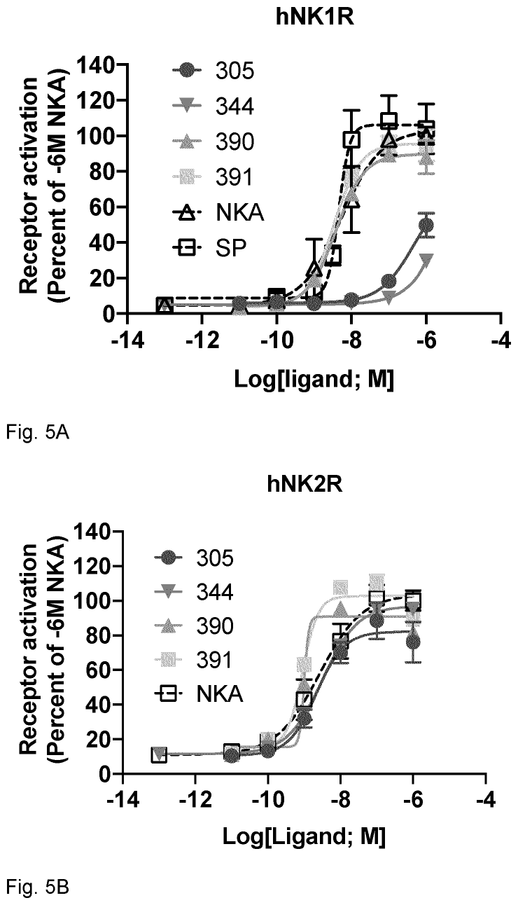

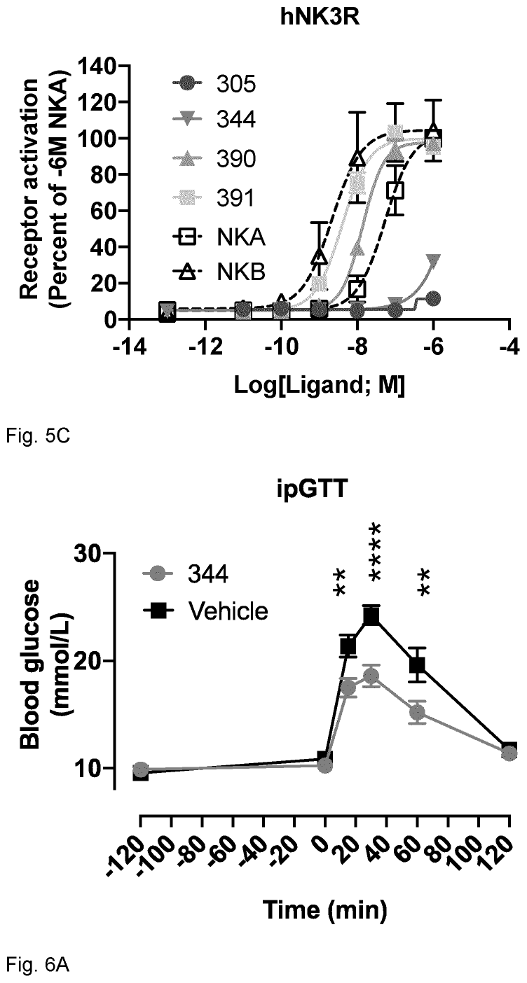

### DETAILED DESCRIPTION

### Terms and Definitions

To facilitate the understanding of the following description, a number of definitions are presented in the following paragraphs.

The term “alkyl” whether used alone or as part of a substituent group, refers to straight and branched carbon chains having 1 to 8 carbon atoms, such as 1 to 6 carbon atoms.

Therefore, designated numbers of carbon atoms (e.g., C₁₋₈) refer independently to the number of carbon atoms in an alkyl moiety or to the alkyl portion of a larger alkyl-containing substituent.

In substituent groups with multiple alkyl groups such as, (C₁₋₆alkyl)₂amino-, the C₁₋₆alkyl groups of the dialkylamino may be the same or different. Alkyl, as defined herein may be substituted by one or more substituents such as a halogen or one or more halogens. In one embodiment, an alkyl is substituted by 1,2 or 3 fluorine atoms.

In one embodiment, an alkyl is substituted by a carboxy group (CO₂), such as a carboxy methyl (CO₂Me).

It is understood that substituents and substitution patterns on the compounds of the present invention can be selected by one of ordinary skill in the art to provide compounds that are chemically stable and that can be readily synthesized by techniques known in the art as well as those methods set forth herein.

The term “subject” refers to an animal, preferably a mammal, and most preferably a human.

Proteinogenic “amino acids” (AA) are named herein using either their 1-letter or 3-letter code according to the recommendations from IUPAC, see for example http-//www.chem.qmul.ac.uk/iupac/AminoAcid/. Capital letter abbreviations indicate L-amino acids, whereas lower case letter abbreviations indicate D-amino acids.

A series of non-proteinogenic amino acids are referred to herein. The names employed should be clear and understandable to the skilled person. meta-Tyrosine (m-Y) is 3-hydroxyphenylalanine. The structure of methoxinine (Mox) is shown below:

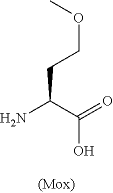

The amino acid, beta-alanine (bA) as used herein is also known as 3-aminopropanoic acid. The amino acid, N-methyl-Leucine is referred to as (NmLeu) herein.

A “terminal fatty acid” is a fatty acid wherein the carboxylic acid group is localized on a terminal carbon atom of the fatty chain. A “terminal C₁₆-C₂₀ fatty acid” is thus a fatty acid chain consisting of 16 to 20 carbon atoms, wherein the acid group is located terminally and the carbon atom of the carboxylic acid group is a terminal chain carbon.

Tachykinin Receptor Activity

Agonist-induced G-protein coupled receptor (GPCR) activation can be measured by an Inositol-1,4,5-Trisphosphate [³H] Radioreceptor Assay (IPs Assay) as described in Example 1. The IP₃ assay takes advantage of the tachykinin receptors' ability to induce production of the inositol trisphosphate (IP₃) second messenger upon agonist (ligand) binding on receptor expressing cells following an initial ³H-inositol labelling period. In effect this means that production of the second messenger IP₃ as a measure of receptor activity can be assessed by counting ³H-activity.

An “NK2R agonist” may possess varying degrees of selectivity relative to activity at the NK1 receptor and/or NK3 receptor as measured in biological assays, such as the IP3 assay presented herein. A “selective NK2R agonist” is herein defined as a ligand that binds to or activates the NK2 receptor with at least about 10 times or greater potency than it binds to or activates the NK1 and/or NK3 receptors. It is not necessary that a molecule be considered selective in both binding and functional (activation) assays to be a selective NK2R agonist. Binding potency is routinely reported as the EC50, with a lower EC50 value equating with greater potency. Thus, a

selective NK2R agonist possesses an NK2R binding EC50 that is at least about 10 times or more lower than its NK1 and/or NK3 binding EC50. Potency to activate a receptor is also routinely reported as the Ki, with the lower Ki value equating with a greater potency.

In a preferred embodiment, the compound provided herein is a neurokinin receptor 2 (NK2R) agonist. In one embodiment, the compound is a selective neurokinin receptor 2 (NK2R) agonist.

In one embodiment, the compound has an EC50 towards human NK2R of 300 nM or less, such as 250 nm or less, such as 200 nm or less, such as 150 nM or less, such as 100 nM or less, such as 90 nM or less, such as 80 nM or less, such as 70 nM or less, such as 60 nM or less, such as 50 nM or less.

In one embodiment, the compound has an EC50 towards human NK2R of 50 nM or less, such as 40 nm or less, such as 30 nm or less, such as 20 nM or less, such as 15 nM or less, such as 14 nM or less, such as 13 nM or less, such as 12 nM or less, such as 11 nM or less, such as 10 nM or less.

In one embodiment, the compound has an EC50 towards human NK1R of at least 100 nM, such as at least 200 nM, such as at least 300 nM, such as at least 400 nM, such as at least 500 nM.

In one embodiment, the compound has an EC50 towards human NK3R of at least 100 nM, such as at least 200 nM, such as at least 300 nM, such as at least 400 nM, such as at least 500 nM.

Tachykinin Receptor Mediated Disorders

In one embodiment, a compound is provided as defined herein for use as a medicament. The present inventors provide the synthesis of chemically stable agonists of NK2R as activators of energy expenditure for treatment of metabolic disorders, such as obesity, as well as for treatment of dysfunctional voiding.

In one embodiment, a method for treating a disease in a subject is provided, comprising administering a compound as herein for treatment of a NK2R mediated disorder.

In one embodiment, the NK2R mediated disorder is selected from the group consisting of: obesity, dysfunctional voiding, diabetes, such as type-II diabetes, and diabetes-related disorders.

In one embodiment, the NK2R mediated disorder is a metabolic disorder. In one embodiment, the metabolic disorder is a diabetes-related disorder. In particular, the diabetes-related disorder is selected from the group consisting of: impaired insulin tolerance and impaired glucose tolerance.

Further, in one embodiment, a method for modulating the activity of NK2R is provided, comprising contacting NK2R with a compound as defined herein.

In one embodiment, a use of a compound as defined herein is provided for the manufacture of a medicament for the treatment of a metabolic disorder.

Peptides—(A)

In a first embodiment, a compound according to formula (I) is provided:

(A)-(B) (I),

wherein;

(A) is a peptide comprising an amino acid sequence of the general formula X₁X₂X₃X₄X₅X₆X₇, wherein
X₁ is selected from the group consisting of: aspartic acid (D) and glutamic acid (E);
X₂ is selected from the group consisting of: lysine (K), arginine (R), and histidine (H);
X₃ is selected from the group consisting of: tyrosine (Y), phenylalanine (F), meta-tyrosine (m-Y), valine (V), tryptophan (W), methionine (M), leucine (L), isoleucine (I), and alanine (A);
X₄ is selected from the group consisting of: valine (V), threonine (T), serine (S), asparagine (N), glutamine (Q), glycine (G), and alanine (A);
X₅ is selected from the group consisting of: glycine (G), 2-aminoisobutyric acid (Aib), serine (S), alanine (A), valine (V), leucine (L), beta-alanine (bA) and isoleucine (I);
X₆ is selected from the group consisting of: leucine (L), isoleucine (I), alanine (A) and N-methyl leucine (Me-Leu); and
X₇ is selected from the group consisting of: norleucine (Nle), methoxinine (Mox), methionine (M), 4-fluorophenylalanine (4fF), and 4-methoxyphenylalanine (4MeOF);

(B) is a conjugated moiety of the general formula (II)

Fa-Lg (II),

wherein;

Fa is a C₁₀-C₂₀ fatty acid, optionally substituted with one or more carboxylic acid groups,

Lg is a linking group, which covalently links (B) to the peptide (A),

and wherein (B) is covalently linked to a terminal amino acid or to a non-terminal amino acid.

In a second embodiment, the compound is provided wherein the peptide (A) is of the general formula X₁X₂X₃X₄X₅X₆X₇, wherein
X₁ is selected from the group consisting of: aspartic acid (D) and glutamic acid (E);
X₂ is selected from the group consisting of: lysine (K), and arginine (R);
X₃ is selected from the group consisting of: tyrosine (Y), and meta-tyrosine (m-Y),
X₄ is selected from the group consisting of: valine (V), and threonine (T);
X₅ is selected from the group consisting of: glycine (G), 2-aminoisobutyric acid (Aib), beta-alanine (bA) and serine (S);
X₆ is selected from the group consisting of: leucine (L), and N-methyl leucine (Me-Leu); and
X₇ is selected from the group consisting of: norleucine (Nle), methoxinine (Mox), methionine (M), 4-fluorophenylalanine (4fF), and 4-methoxyphenylalanine (4MeOF).

In a third embodiment, the compound is provided wherein the peptide (A) is of the general formula X₁X₂X₃X₄X₅X₆X₇, wherein
X₁ is selected from the group consisting of: aspartic acid (D) and glutamic acid (E);
X₂ is selected from the group consisting of: lysine (K), and arginine (R);
X₃ is selected from the group consisting of: tyrosine (Y), and phenylalanine (F), and meta-tyrosine (m-Y),
X₄ is selected from the group consisting of: valine (V), and threonine (T);
X₅ is selected from the group consisting of: glycine (G), 2-aminoisobutyric acid (Aib), beta-alanine (bA) and serine (S);
X₆ is selected from the group consisting of: leucine (L), and N-methyl leucine (Me-Leu); and
X₇ is selected from the group consisting of: norleucine (Nle), methoxinine (Mox), methionine (M), 4-fluorophenylalanine (4fF), and 4-methoxyphenylalanine (4MeOF).

In one embodiment, the compound is provided wherein X₂ is arginine (R).

In one embodiment, the compound is provided wherein X₃ is tyrosine (Y). In one embodiment, the compound is provided wherein X₃ is tyrosine (Y) and wherein X₂ is arginine (R). In one embodiment, X₃ is tyrosine (Y), X₂ is arginine (R), and X₅ is 2-aminoisobutyric acid (Aib).

In one embodiment, the compound is provided wherein X₄ is threonine (T).

In one embodiment, the compound is provided wherein X₅ is selected from the group consisting of: 2-aminoisobutyric acid (Aib) and serine (S).

In one embodiment, the compound is provided wherein X₆ is N-methyl-leucine (Me-Leu).

In one embodiment, the compound is provided wherein X₇ is methoxinine (Mox). In one embodiment, the compound is provided wherein X₇ is methoxinine (Mox) and wherein X₂ is arginine (R). In one embodiment, X₇ is methoxinine (MOx), X₂ is arginine (R), and X₃ is tyrosine (Y).

Preferably, the peptide (A) is amidated on the C-terminus.

In one embodiment, the peptide (A) comprises from 7 to 15 amino acids, such as from 7 to 14 amino acids, such as from 7 to 13 amino acids, such as from 7 to 12 amino acids, such as from 7 to 11 amino acids, such as from 7 to 11 amino acids, such as from 7 to 10 amino acids, such as from 7 to 9 amino acids, such as from 7 to 8 amino acids, preferably wherein the peptide comprises 7 amino acids.

In one embodiment, the peptide (A) comprises no more than 15 amino acids, such as no more than 14 amino acids, such as no more than 13 amino acids, such as no more than 12 amino acids, such as no more than 11 amino acids, such as no more than 10 amino acids, such as no more than 9 amino acids, such as no more than 8 amino acids, such as no more than 7 amino acids.

In one embodiment, the peptide (A) consists of 7 amino acids of the general formula X₁X₂X₃X₄X₅X₆X₇. The peptide is preferably amidated on the C-terminus.

In one particular embodiment, the compound is provided wherein
(A) is: Asp;Lys;Phe;Val;Gly;NmLeu;Nle;NH2 (compound 305), and
(B) is of formula (B1) covalently attached to the N-terminal asparagine of (A).

In one particular embodiment, the compound is provided wherein
(A) is: Asp;Lys;Tyr;Val;Gly;NmLeu;Metox;NH2 (compound 344), and
(B) is of formula (B1) covalently attached to the N-terminal aspartate of (A).

In one embodiment, the compound consists of the sequence of any one of SEQ ID NO: 1 to SEQ ID NO: 57.

In one particular embodiment, the compound is:

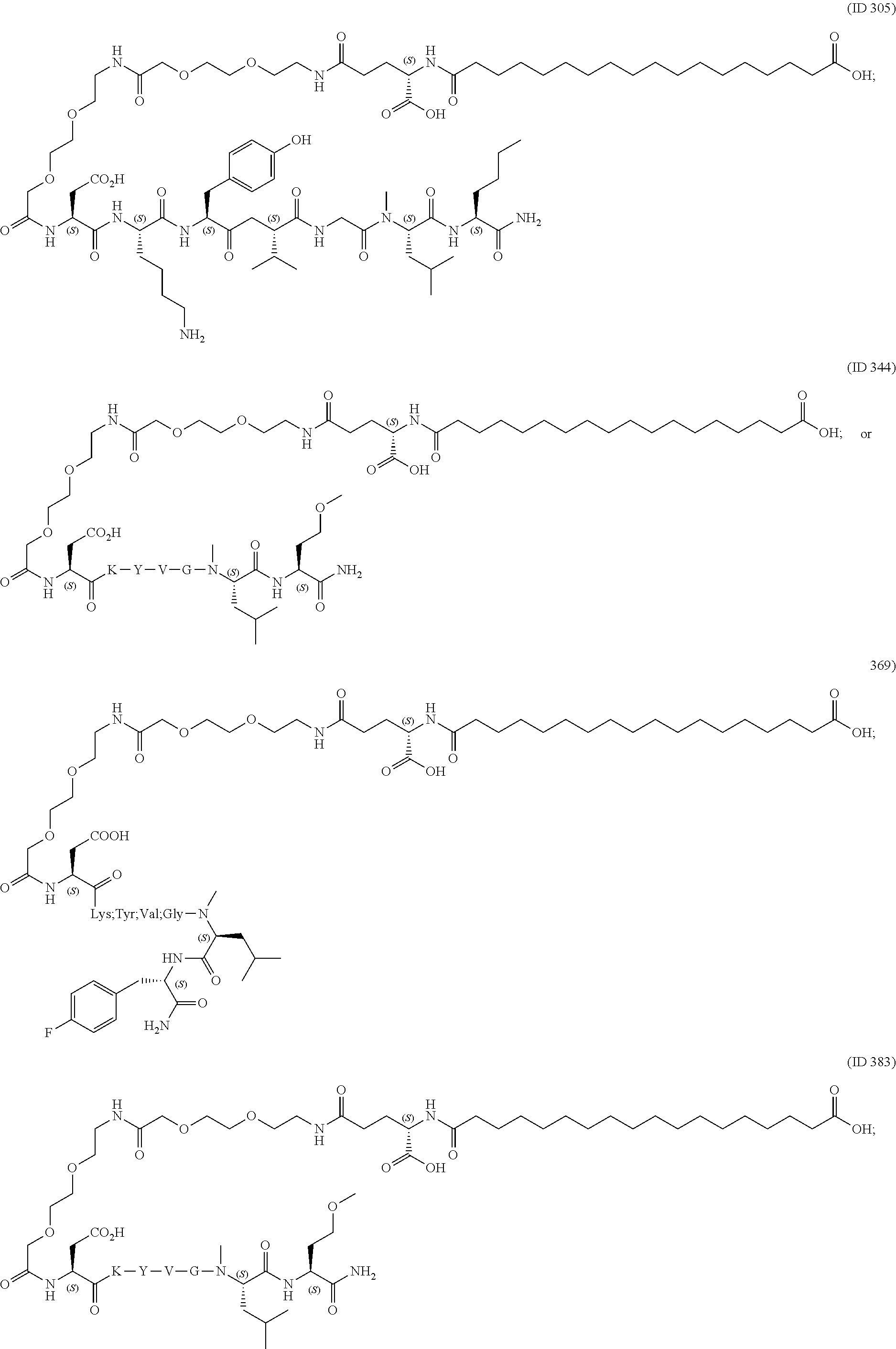

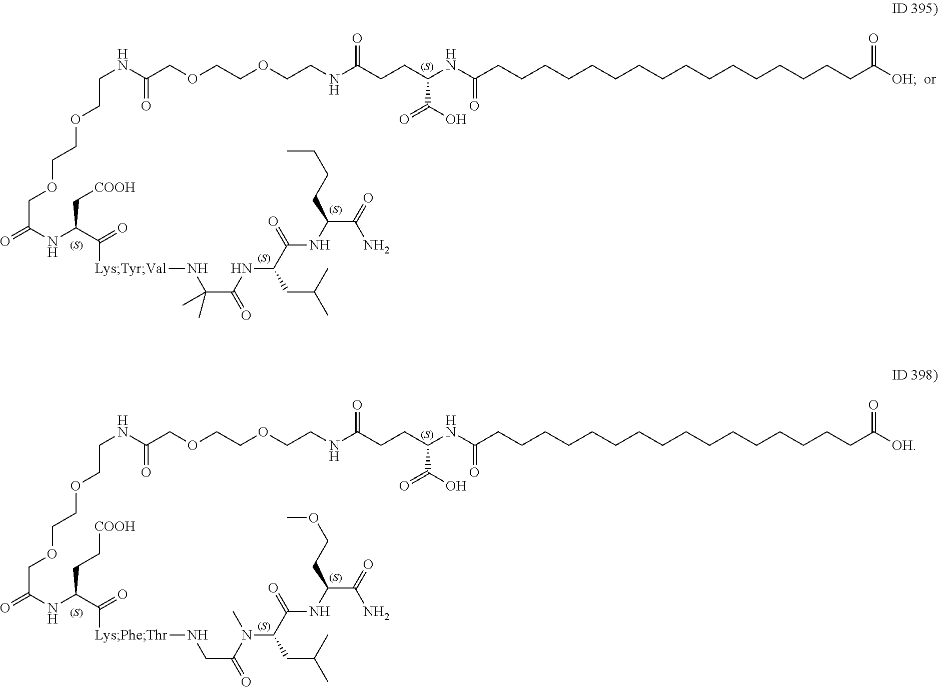

In the context of the present disclosure and unless otherwise stated, when a bond is drawn from an atom to an amino acid abbreviated by its one or three letter code, the bond is connected to the α-amino group or to the carbonyl carbon of the amino acid's backbone. For example in the case of compound with ID 398, “Lys” is connected to its adjacent carbonyl via its α-amino group, and “Thr” is connected to “NH” via its backbone carbonyl carbon.

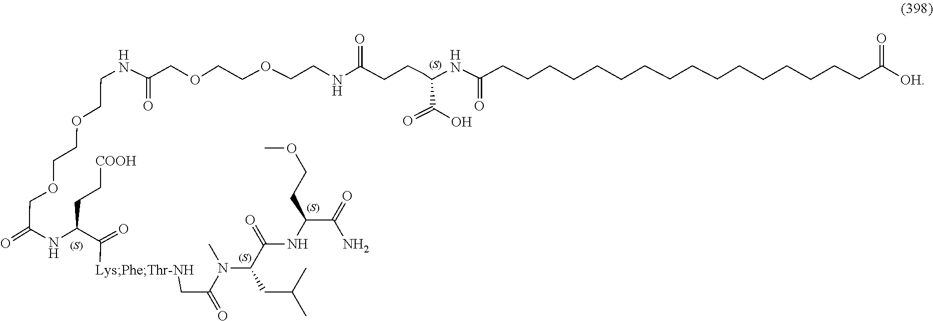

Conjugated Moieties—(B)

Conjugated moieties are also referred to as protractors herein. In one embodiment, the compound as defined herein is provided, wherein Lg is of formula (Lg-1),

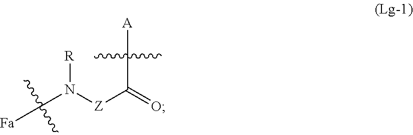

wherein Z is a chain comprising from 18 to 23 atoms in the backbone selected from the group consisting of: C, O, and N;

and wherein R is selected from the group consisting of H, and C₁₋₆ alkyl. A person of skill in the art knows that C, O, and N may be substituted with hydrogen according to the valence of the particular atom. The backbone may comprise one or more carbonyl groups, such as 1, 2, 3, or 4 carbonyl groups. The backbone may also comprise one or more carboxylic acid groups, such as 1 or 2. In some embodiments, Z comprises fragments of ethylene glycol interrupted by one or more amide functionalities. An example is shown in formula (B1), wherein the fatty acid Fa is of formula (Fa-1), R of Lg-1 is H, and the backbone of Z comprises 21 atoms selected from the group consisting of C, O, and N.

In one embodiment, the compound is provided, wherein Fa is a terminal C₁₆-C₂₀ fatty acid.

In one embodiment, the compound is provided wherein Fa is of formula (Fa-1),

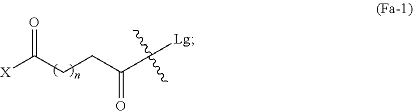

wherein n is from 11 to 20, such as from 12 to 19, for example from 13 to 18, such as from 14 to 17, preferably wherein n is 15;

and wherein X is selected from the group consisting of —OH, —OC₁₋₆, —NH₂, —NHC₁₋₆, and N(C₁₋₆)₂. In a particular embodiment, n is 15 and X is —OH.

In one embodiment, the compound is provided wherein Lg of the conjugated moiety does not comprise functional groups that are positively charged at pH=7.4. In one embodiment, Lg of the conjugated moiety does not comprise more than 1 functional group that is negatively charged at pH=7.4. In one embodiment, Lg of the conjugated moiety has a net neutral charge or −1 at pH=7.4.

In one preferred embodiment, the compound as defined herein is provided, wherein the conjugated moiety is of formula (B1);

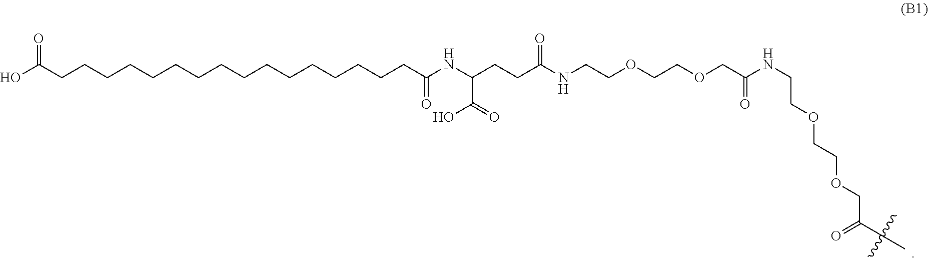 [SMILES: *C(=O)COCCOCCNC(=O)COCCOCCNC(=O)CCC(NC(=O)CCCCCCCCCCCCCCCCC(=O)O)C(=O)O]

In a particularly preferred embodiment, the compound as defined herein is provided wherein the conjugated moiety is of the formula below;

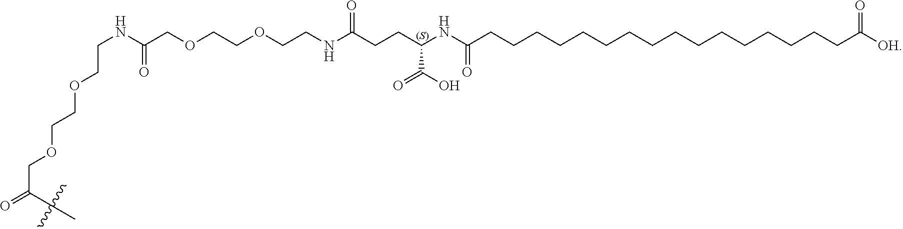

In one embodiment, the conjugated moiety (B) is covalently attached to the N-terminus of (A), optionally via an amide bond.

In one embodiment, the conjugated moiety (B) is covalently attached to the N-terminus of (A) via an amide bond with the N-terminal α-NH₂ group.

Pharmaceutical Compositions

In one embodiment, a pharmaceutical composition is provided comprising the compound as defined herein, and one or more pharmaceutically acceptable adjuvants, excipients, carriers, buffers and/or diluents.

Items

1. A compound according to formula (I):

(A)-(B) (I),
wherein;
(A) is a peptide comprising an amino acid sequence of the general formula X₁X₂X₃X₄X₅X₆X₇, wherein
X₁ is selected from the group consisting of: aspartic acid (D) and glutamic acid (E);
X₂ is selected from the group consisting of: lysine (K), arginine (R), and histidine (H);
X₃ is selected from the group consisting of: tyrosine (Y), phenylalanine (F), meta-tyrosine (m-Y), valine (V), tryptophan (W), methionine (M), leucine (L), isoleucine (I), and alanine (A);
X₄ is selected from the group consisting of: valine (V), threonine (T), serine (S), asparagine (N), glutamine (Q), glycine (G), and alanine (A);
X₅ is selected from the group consisting of: glycine (G), 2-aminoisobutyric acid (Aib), serine (S), alanine (A), valine (V), leuicine (L), beta-alanine (bA) and isoleucine (I);
X₆ is selected from the group consisting of: leucine (L), isoleucine (I), alanine (A) and N-methyl leucine (Me-Leu); and
X₇ is selected from the group consisting of: norleucine (Nle), methoxinine (Mox), methionine (M), 4-fluorophenylalanine (4fF), and 4-methoxyphenylalanine (4MeOF);
(B) is a conjugated moiety of the general formula (II)

Fa-Lg (II),
wherein;
Fa is a C₁₀-C₂₀ fatty acid, optionally substituted with one or more carboxylic acid groups,
Lg is a linking group, which covalently links (B) to the peptide (A),
and wherein (B) is covalently linked to a terminal amino acid or to a non-terminal amino acid.
   2. The compound according to any one of the preceding items, wherein the peptide (A) is of the general formula X₁X₂X₃X₄X₅X₆X₇, wherein
X₁ is selected from the group consisting of: aspartic acid (D) and glutamic acid (E);
X₂ is selected from the group consisting of: lysine (K), and arginine (R);
X₃ is selected from the group consisting of: tyrosine (Y), and phenylalanine (F), and meta-tyrosine (m-Y),
X₄ is selected from the group consisting of: valine (V), and threonine (T);
X₅ is selected from the group consisting of: glycine (G), 2-aminoisobutyric acid (Aib), beta-alanine (bA) and serine (S);
X₆ is selected from the group consisting of: leucine (L), and N-methyl leucine (Me-Leu); and
X₇ is selected from the group consisting of: norleucine (Nle), methoxinine (Mox), methionine (M), 4-fluorophenylalanine (4fF), and 4-methoxyphenylalanine (4MeOF).
   3. The compound according to any one of the preceding items, wherein X₂ is arginine (R).
   4. The compound according to any one of the preceding items, wherein X₃ is tyrosine (Y).
   5. The compound according to any one of the preceding items, wherein X₄ is threonine (T).
   6. The compound according to any one of the preceding items, wherein X₅ is selected from the group consisting of: 2-aminoisobutyric acid (Aib) and serine (S).
   7. The compound according to any one of the preceding items, wherein X₆ is N-methyl-leucine (Me-Leu).
   8. The compound according to any one of the preceding items, wherein X₇ is methoxinine (Mox).
   9. The compound according to any one of the preceding items, wherein Lg is of formula (Lg-1),

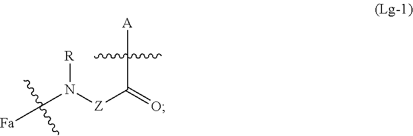

wherein Z is a chain comprising from 18 to 23 atoms in the backbone selected from the group consisting of: C, O, and N;
and wherein R is selected from the group consisting of H, and C₁₋₆ alkyl.
   10. The compound according to any one of the preceding items, wherein Fa is a terminal C₁₆-C₂₀ fatty acid.
   11. The compound according to any one of the preceding items, wherein Fa is of formula (Fa-1),

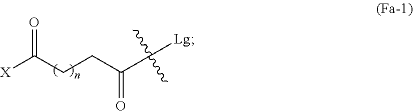

wherein n is from 11 to 20, such as from 12 to 19, for example from 13 to 18, such as from 14 to 17, preferably wherein n is 15;
and wherein X is selected from the group consisting of —OH, —OC₁₋₆, —NH₂, —NHC₁₋₆, and N(C₁₋₆)₂.
   12. The compound according to item 11, wherein n is 15 and wherein X is —OH.
   13. The compound according to any one of the preceding items, wherein Lg of the conjugated moiety does not comprise functional groups that are positively charged at pH=7.4.
   14. The compound according to any one of the preceding items, wherein Lg of the conjugated moiety has a net neutral charge or −1 at pH=7.4.
   15. The compound according to any one of the preceding items, wherein the conjugated moiety is of formula (B1);

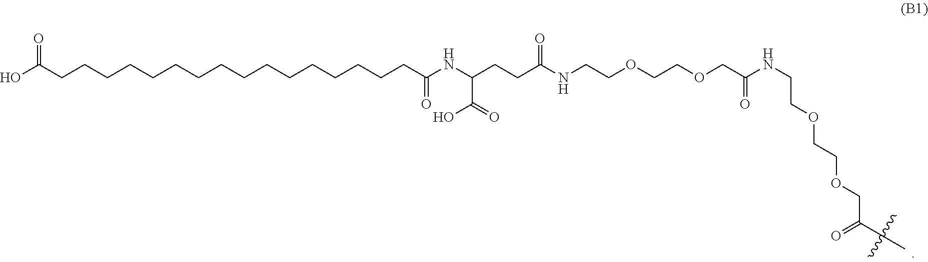 [SMILES: *C(=O)COCCOCCNC(=O)COCCOCCNC(=O)CCC(NC(=O)CCCCCCCCCCCCCCCCC(=O)O)C(=O)O]

16. The compound according to any one of the preceding items, wherein the conjugated moiety (B) is covalently attached to the N-terminus of (A), optionally via an amide bond.
17. The compound according to any one of the preceding items, wherein the conjugated moiety (B) is covalently attached to the N-terminus of (A) via an amide bond with the N-terminal α-NH₂ group.
18. The compound according to any one of the preceding items, wherein the peptide (A) is amidated on the C-terminus.
19. The compound according to any one of the preceding items, wherein the peptide (A) comprises from 7 to 15 amino acids, such as from 7 to 14 amino acids, such as from 7 to 13 amino acids, such as from 7 to 12 amino acids, such as from 7 to 11 amino acids, such as from 7 to 11 amino acids, such as from 7 to 10 amino acids, such as from 7 to 9 amino acids, such as from 7 to 8 amino acids, preferably wherein the peptide comprises 7 amino acids.
20. The compound according to any one of the preceding items, wherein the peptide (A) comprises no more than 15 amino acids, such as no more than 14 amino acids, such as no more than 13 amino acids, such as no more than 12 amino acids, such as no more than 11 amino acids, such as no more than 10 amino acids, such as no more than 9 amino acids, such as no more than 8 amino acids, such as no more than 7 amino acids.
21. The compound according to any one of the preceding items, wherein the peptide (A) consists of 7 amino acids of the general formula X₁X₂X₃X₄X₅X₆X₇.
22. The compound according to any one of the preceding items, wherein
(A) is: Asp;Lys;Phe;Val;Gly;NmLeu;Nle;NH2 (compound 305), and
(B) is of formula (B1) covalently attached to the N-terminal aspartate of (A).
   23. The compound according to any one of the preceding items, wherein
(A) is: Asp;Lys;Tyr;Val;Gly;NmLeu;Metox;NH2 (compound 344), and
(B) is of formula (B1) covalently attached to the N-terminal aspartate of (A).
   24. The compound according to any one of the preceding items, wherein the compound consists of the sequence of any one of SEQ ID NO: 1 to SEQ ID NO: 57.
   25. The compound according to any one of the preceding items, wherein the compound is:

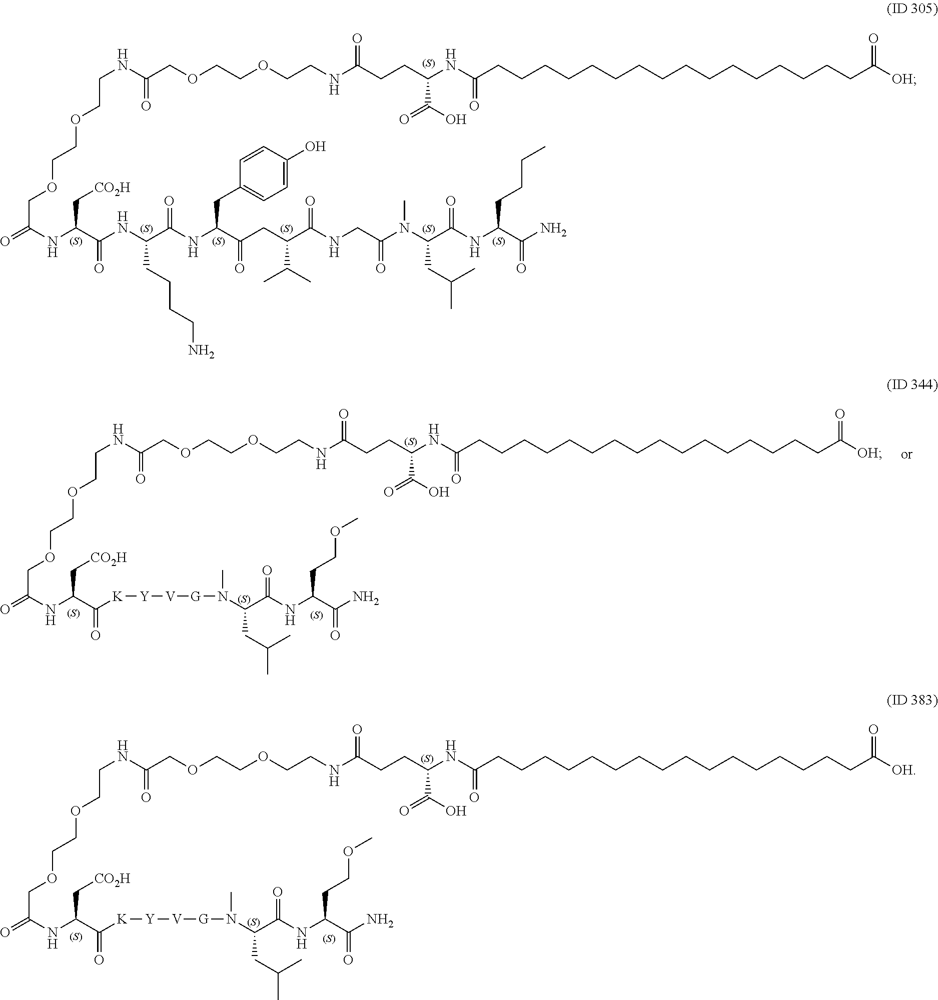

26. The compound according to any one of the preceding items, wherein the compound is a neurokinin receptor 2 (NK2R) agonist.
27. The compound according to any one of the preceding items, wherein the compound is a selective neurokinin receptor 2 (NK2R) agonist.
28. The compound according to any one of the preceding items, wherein the compound has an EC50 towards human NK2R of 300 nM or less, such as 250 nm or less, such as 200 nm or less, such as 150 nM or less, such as 100 nM or less, such as 90 nM or less, such as 80 nM or less, such as 70 nM or less, such as 60 nM or less, such as 50 nM or less.
29. The compound according to any one of the preceding items, wherein the compound has an EC50 towards human NK2R of 50 nM or less, such as 40 nm or less, such as 30 nm or less, such as 20 nM or less, such as 15 nM or less, such as 14 nM or less, such as 13 nM or less, such as 12 nM or less, such as 11 nM or less, such as 10 nM or less.
30. The compound according to any one of the preceding items, wherein the compound has an EC50 towards human NK1R of at least 100 nM, such as at least 200 nM, such as at least 300 nM, such as at least 400 nM, such as at least 500 nM.
31. The compound according to any one of the preceding items, wherein the compound has an EC50 towards human NK3R of at least 100 nM, such as at least 200 nM, such as at least 300 nM, such as at least 400 nM, such as at least 500 nM.
32. A pharmaceutical composition comprising the compound as defined in any one of the preceding items, and one or more pharmaceutically acceptable adjuvants, excipients, carriers, buffers and/or diluents.
33. A compound as defined in any one items 1 to 31 for use as a medicament.
34. A method for treating a disease in a subject comprising administering a compound as defined in any one items 1 to 31 for treatment of a NK2R mediated disorder.
35. The method according to any one of the preceding items, wherein the NK2R mediated disorder is selected from the group consisting of: obesity, dysfunctional voiding, diabetes, such as type-II diabetes, and diabetes-related disorders.
36. The method according to any one of the preceding items, wherein the NK2R mediated disorder is a metabolic disorder.
37. The method according to any one of the preceding items, wherein the metabolic disorder is a diabetes-related disorder.
38. The method according to item 35, wherein the diabetes-related disorder is selected from the group consisting of: impaired insulin tolerance and impaired glucose tolerance.
39. A method for modulating the activity of NK2R, comprising contacting NK2R with a compound as defined in any one items 1 to 31.
40. Use of a compound as defined in any one items 1 to 31 for the manufacture of a medicament for the treatment of a metabolic disorder.

Items II

1. A compound according to formula (I):

(A)-(B) (I),
wherein:
(A) is a peptide comprising an amino acid sequence of the general formula X₁X₂X₃X₄X₅X₆X₇, wherein
X₁ is selected from the group consisting of: aspartic acid (D) and glutamic acid (E);
X₂ is selected from the group consisting of: lysine (K), arginine (R), and histidine (H);
X₃ is selected from the group consisting of: tyrosine (Y), phenylalanine (F), meta-tyrosine (m-Y), valine (V), tryptophan (W), methionine (M), leucine (L), isoleucine (I), and alanine (A);
X₄ is selected from the group consisting of: valine (V), threonine (T), serine (S), asparagine (N), glutamine (Q), glycine (G), and alanine (A);
X₅ is selected from the group consisting of: glycine (G), 2-aminoisobutyric acid (Aib), serine (S), alanine (A), valine (V), leuicine (L), beta-alanine (bA) and isoleucine (I);
X₆ is selected from the group consisting of: leucine (L), isoleucine (I), alanine (A) and N-methyl leucine (Me-Leu); and
X₇ is selected from the group consisting of: norleucine (Nle), methoxinine (Mox), methionine (M), 4-fluorophenylalanine (4fF), and 4-methoxyphenylalanine (4MeOF);
(B) is a conjugated moiety of the general formula (II)

Fa-Lg (II),
wherein;
Fa is a C₁₀-C₂₀ fatty acid, optionally substituted with one or more carboxylic acid groups,
Lg is a linking group, which covalently links (B) to the peptide (A),
and wherein (B) is covalently linked to a terminal amino acid or to a non-terminal amino acid.
   2. The compound according to any one of the preceding items, wherein the peptide (A) is of the general formula X₁X₂X₃X₄X₅X₆X₇, wherein
X₁ is selected from the group consisting of: aspartic acid (D) and glutamic acid (E);
X₂ is selected from the group consisting of: lysine (K), and arginine (R);
X₃ is selected from the group consisting of: tyrosine (Y), and phenylalanine (F), and meta-tyrosine (m-Y),
X₄ is selected from the group consisting of: valine (V), and threonine (T);
X₅ is selected from the group consisting of: glycine (G), 2-aminoisobutyric acid (Aib), beta-alanine (bA) and serine (S);
X₆ is selected from the group consisting of: leucine (L), and N-methyl leucine (Me-Leu); and
X₇ is selected from the group consisting of: norleucine (Nle), methoxinine (Mox), methionine (M), 4-fluorophenylalanine (4fF), and 4-methoxyphenylalanine (4MeOF).
   3. The compound according to any one of the preceding items, wherein
X₂ is arginine (R);
X₃ is tyrosine (Y);
X₄ is threonine (T);
X₅ is selected from the group consisting of: 2-aminoisobutyric acid (Aib) and serine (S);
X₆ is N-methyl-leucine (Me-Leu); and/or
X₇ is methoxinine (Mox).
   4. The compound according to any one of the preceding items, wherein Lg is of formula (Lg-1),

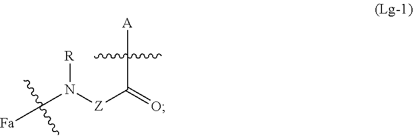

wherein Z is a chain comprising from 18 to 23 atoms in the backbone selected from the group consisting of: C, O, and N;
and wherein R is selected from the group consisting of H, and C₁₋₆ alkyl.
   5. The compound according to any one of the preceding items, wherein Fa is a terminal C₁₆-C₂₀ fatty acid.
   6. The compound according to any one of the preceding items, wherein Fa is of formula (Fa-1),

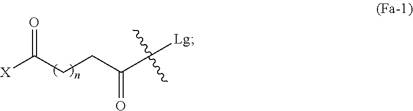

wherein n is from 11 to 20, such as from 12 to 19, for example from 13 to 18, such as from 14 to 17, preferably wherein n is 15;
and wherein X is selected from the group consisting of —OH, —OC₁₋₆, —NH₂, —NHC₁₋₆, and N(C₁₋₆)₂.
   7. The compound according to any one of the preceding items, wherein the conjugated moiety is of formula (B1);

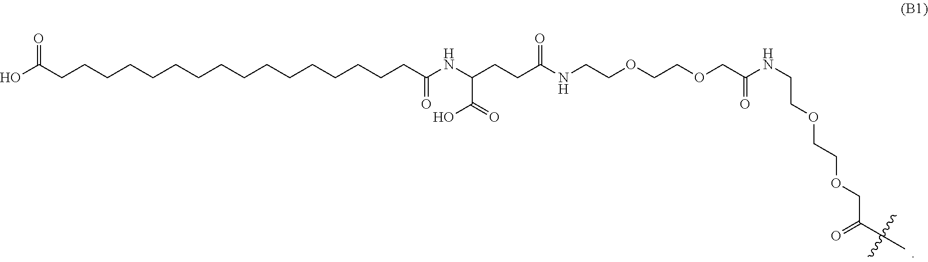 [SMILES: *C(=O)COCCOCCNC(=O)COCCOCCNC(=O)CCC(NC(=O)CCCCCCCCCCCCCCCCC(=O)O)C(=O)O]

8. The compound according to any one of the preceding items, wherein the conjugated moiety (B) is covalently attached to the N-terminus of (A) via an amide bond with the N-terminal α-NH₂ group.
9. The compound according to any one of the preceding items, wherein the peptide (A) is amidated on the C-terminus.
10. The compound according to any one of the preceding items, wherein the peptide (A) comprises from 7 to 15 amino acids, such as from 7 to 14 amino acids, such as from 7 to 13 amino acids, such as from 7 to 12 amino acids, such as from 7 to 11 amino acids, such as from 7 to 11 amino acids, such as from 7 to 10 amino acids, such as from 7 to 9 amino acids, such as from 7 to 8 amino acids, preferably wherein the peptide comprises 7 amino acids.
11. The compound according to any one of the preceding items, wherein the peptide (A) consists of 7 amino acids of the general formula X₁X₂X₃X₄X₅X₆X₇.
12. The compound according to any one of the preceding items, wherein the compound consists of the sequence of any one of SEQ ID NO: 1 to SEQ ID NO: 57.
13. The compound according to any one of the preceding items, wherein
(A) is: Asp;Lys;Tyr;Val;Gly;NmLeu;Metox;NH2 (compound 344), and
(B) is of formula (B1) covalently attached to the N-terminal aspartate of (A).
   14. The compound according to any one of the preceding items, wherein the compound is:

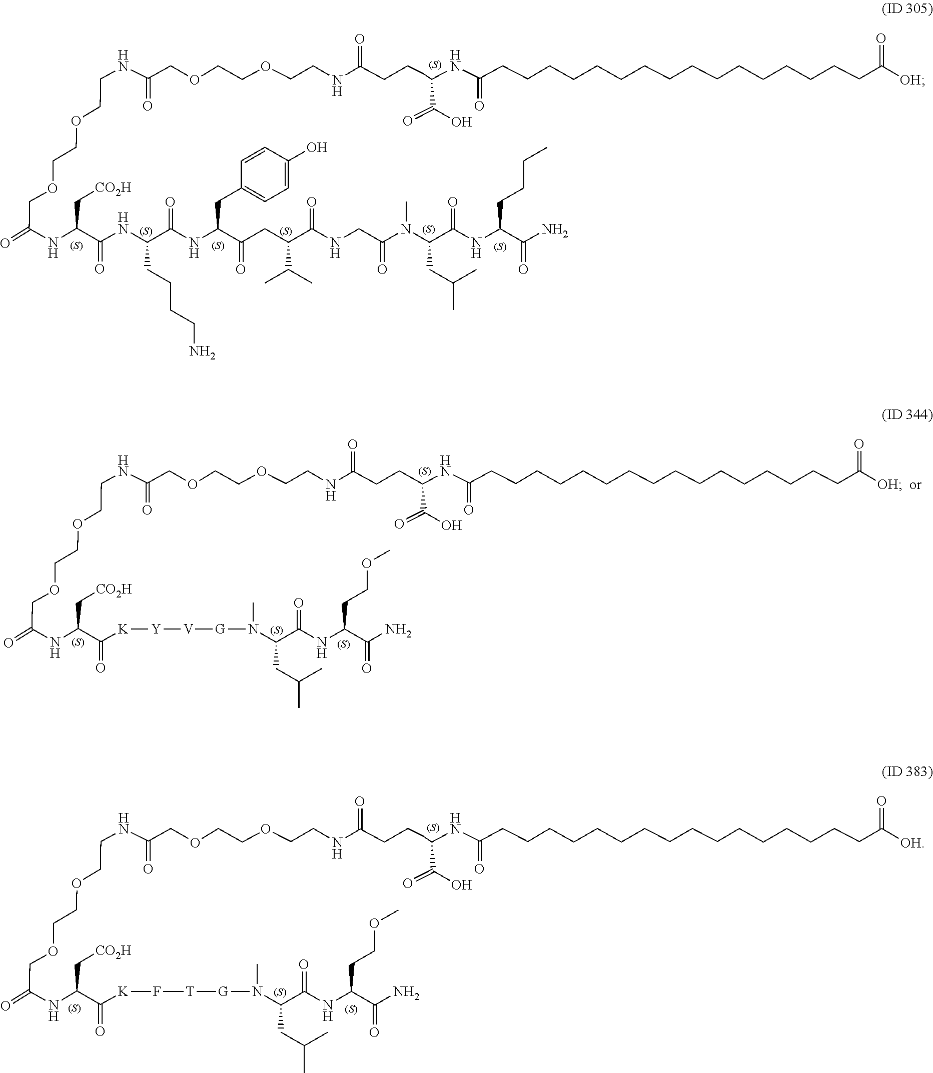

15. The compound according to any one of the preceding items, wherein the compound is a neurokinin receptor 2 (NK2R) agonist, such as a selective neurokinin receptor 2 (NK2R) agonist.

### EXAMPLES

### Example 1: Determination of Compound Selectivity Through Measuring Potencies and Efficacies by Inositol Trisphosphate (IP9) Measurement

Materials:

Formic acid, LiCl, CaCl₂, Tris-HCl, EDTA, HEPES, NaCl, Chloroquine, and ovalbumin (albumin from chicken eggs) (Sigma Aldrich). COS-7 monkey kidney cell line was obtained from ATCC. DMEM 1885, FBS, Penicillin/Streptomycin (P/S) and HBSS were from Thermo Scientific/Gibco. Clear Costar 96 wells Tissue Culture-treated plates and solid white 96-well plates from Corning. Polylysine Coated Yttrium Silicate SPA Beads (#RPNQ0010) and Myo-[2-³H(N)]-inositol—(#NET114A[005MC]) from Perkin Elmer. Inositol-1,4,5-Trisphosphate [³H] Radioreceptor Assay (IPs Assay) from Perkin Elmer. pcDNA3.1(+) containing coding sequences of human and mouse tachykinin receptor 1, 2, 3 mRNA were obtained from Genscript (custom order). Synthesized peptides diluted in saline+0.2% (w/v) ovalbumin.

mRNA IDs
Human Tacr1: NM_001058.4
Human Tacr2: NM_001057.3
Human Tacr3: NM_001059.2

Methods:

Agonist-induced G-protein coupled receptor (GPCR) activation was measured by an Inositol-1,4,5-Trisphosphate [³H] Radioreceptor Assay (IPs Assay). Assays were carried out using COS-7 cells transiently transfected by calcium phosphate transfection with a vector pcDNA3.1(+) encoding one of the indicated receptors (Genscript). Briefly, DNA mixed with CaCl₂ (2 M) and TE-buffer (10 mM Tris-HCl, 1 mM EDTA, pH 7.5) was dropwise added to 2×HBS (50 mM HEPES, 280 mM NaCl, 1.5 mM NaH₂PO₄, pH 7.2) and incubated for 45 min at room temperature (22±2° C.). The mixture and a final concentration of 100 μM Chloroquine were added to the cells and left to incubate for 5 hours at 37° C. under standard cell culture conditions (10% CO₂) before changing medium to fresh medium containing 5 μl/mL Myo-[2-³H(N)]-inositol (labelling medium).

The IP₃ assay takes advantage of the tachykinin receptors' ability to induce production of the inositol trisphosphate (IP₃) second messenger upon agonist (ligand) binding on receptor expressing cells following an initial ³H-inositol labelling period. In effect this means that production of the second messenger IP₃ as a measure of receptor activity can be assessed by counting ³H-activity.

Assay solutions used: Wash buffer (HBSS), Assay buffer (HBSS+10 mM LiCl and 0,2% w/v ovalbumin), Lysis buffer (10 mM formic acid), and SPA YSI beads (12.5 mg/ml in H₂O). The assay was performed the day after transfection. Briefly, labelling medium was aspirated, and plates were washed ×1 in wash buffer before adding 100 μl assay buffer, pre-incubated for 30 min followed by 120 min incubation with agonist, both at 37° C. After incubation, plates were immediately placed on ice and the incubation medium was aspirated and 40 μl of 10 mM formic acid per well was added. Plates were incubated for at least 30 min on ice. 60 μl (1 mg/well) SPA YSI beads/well was pipetted into a solid white 96 wells plate and 35 μl of the lysis solution was transferred to the plate before covering plates with seal cover and shaking for 10 min. (max speed). Centrifuge plates and leave for 8 hours at room temperature before counting the plates in a MicroBeta plate counter (Perkin Elmer).

### Example 2: Determination of Human Serum Albumin (HSA) Binding by Measuring Receptor Activity Using Inositol Trisphosphate (IP9) Quantification

Materials:

Formic acid, LiCl, CaCl₂, Tris-HCl, EDTA, HEPES, NaCl, NaH₂PO₄, Chloroquine, ovalbumin (albumin from chicken eggs), and human serum albumin (HSA) (Sigma Aldrich). COS-7 monkey kidney cell line was obtained from ATCC. DMEM 1885, FBS, Penicillin/Streptomycin (P/S) and HBSS were from Thermo Scientific/Gibco. Clear Costar 96 wells Tissue Culture-treated plates and solid white 96-well plates from Corning. Polylysine Coated Yttrium Silicate SPA Beads (#RPNQ0010) and Myo-[2-³H(N)]-inositol—(#NET114A[005MC]) from Perkin Elmer. Inositol-1,4,5-Trisphosphate [³H] Radioreceptor Assay (IPs Assay) from Perkin Elmer. pcDNA3.1(+) containing coding sequences of human tachykinin receptor 1, 2, 3 mRNA were obtained from Genscript (custom order). Synthesized peptides diluted in saline+0.2% (w/v) ovalbumin or saline+1% (w/v) HSA.
Human Tacr1: NM_001058.4
Human Tacr2: NM_001057.3
Human Tacr3: NM_001059.2

Methods:

Agonist-induced G-protein coupled receptor (GPCR) activation was measured by an Inositol-1,4,5-Trisphosphate [³H] Radioreceptor Assay (IPs Assay). Assays were carried out using COS-7 cells transiently transfected by calcium phosphate transfection with a vector pcDNA3.1(+) encoding one of the indicated receptors (Genscript). Briefly, DNA mixed with CaCl₂ (2 M) and TE-buffer (10 mM Tris-HCl, 1 mM EDTA, pH 7.5) was dropwise added to 2×HBS (50 mM HEPES, 280 mM NaCl, 1.5 mM NaH₂PO₄, pH 7.2) and incubated for 45 min at room temperature (22±2° C.). The mixture and a final concentration of 100 μM Chloroquine were added to the cells and left to incubate for 5 hours at 37° C. under standard cell culture conditions (10% CO₂) before changing medium to fresh medium containing 5 μl/mL Myo-[2-³H(N)]-inositol (labeling medium).

The indirect HSA binding IP₃ assay takes advantage of the tachykinin receptors' ability to induce production of the inositol trisphosphate (IP₃) second messenger upon agonist (ligand) binding on receptor expressing cells following an initial ³H-inositol labeling period. The assay relies on the assumption that high peptide HSA binding will result in low receptor-mediated production of the second messenger IP₃. Thus, the assay is an indirect assessment HSA binding.

Assay solutions used: Wash buffer (HBSS), Assay buffer 0.2% OvAlb (HBSS+10 mM LiCl and 0,2% w/v ovalbumin) or Assay buffer 1% HSA (HBSS+10 mM LiCl and 1% w/v HSA), Lysis buffer (10 mM formic acid), and SPA YSI beads (12.5 mg/ml in H₂O). The assay was performed the day after transfection. Briefly, labelling medium was aspirated, and plates were washed ×1 in wash buffer before adding 100 μl assay buffer 0.2% OvAlb or assay buffer 1% HSA, pre-incubated for 30 min followed by 120 min incubation with agonist, both at 37° C. After incubation, plates were immediately placed on ice and the incubation medium was aspirated and 40 μl of 10 mM formic acid per well was added. Plates were incubated for at least 30 min on ice. 60 μl (1 mg/well) SPA YSI beads/well was pipetted into a solid white 96 wells plate and 35 μl of the lysis solution was transferred to the plate before covering plates with seal cover and shaking for 10 min. (max speed). Centrifuge plates and leave for 8 hours at room temperature before counting the plates in a MicroBeta plate counter (Perkin Elmer).

### Example 3: Determination of Peptide-NK2R Binding by Measuring ³H-NKA Competitive Binding

Materials:

CaCl₂, Tris-HCl, EDTA, HEPES, NaCl, NaH₂PO₄, Chloroquine, ovalbumin (albumin from chicken eggs, MnCl₂·4H₂O, and Bacitracin from Sigma Aldrich. COS-7 monkey kidney cell line was obtained from ATCC. DMEM 1885, FBS, Penicillin/Streptomycin (P/S), HBSS, and 1M Tris/HCl were from Thermo Scientific/Gibco. White/Clear bottom 96 well plates from Costar. ³H-NKA (Novo Nordisk #NNC0392-0000-0497). Ultima Gold XR from Perkin Elmer.

pcDNA3.1(+) containing coding sequences of human and mouse tachykinin receptor 1, 2, 3 mRNA were obtained from Genscript (custom order). Synthesized peptides diluted in saline+0.2% (w/v) ovalbumin (OvAlb).
Human Tacr1: NM_001058.4
Human Tacr2: NM_001057.3
Human Tacr3: NM_001059.2

Methods:

Assays were carried out using COS-7 cells transiently transfected by calcium phosphate transfection with a vector pcDNA3.1(+) encoding one of the indicated receptors (Genscript). Briefly, DNA mixed with CaCl₂ (2 M) and TE-buffer (10 mM Tris-HCl, 1 mM EDTA, pH 7.5) was dropwise added to 2×HBS (50 mM HEPES, 280 mM NaCl, 1.5 mM NaH₂PO₄, pH 7.2) and incubated for 45 min at room temperature (22±2° C.). The mixture and a final concentration of 100 μM Chloroquine were added to the cells and left to incubate for 5 hours at 37° C. under standard cell culture conditions (10% CO₂) before changing medium to fresh maintenance medium.

The ³H-NKA binding assay measures peptide-receptor binding by a competitive principle of receptor binding between radioactively labelled (3H) NKA (tracer) and synthesized peptide ligands on live cells expressing the receptor of interest. Assay solutions used: TKR buffer (50 mM Tris/HCl pH 7.5, 5 mM MnCl₂, and 150 mM NaCl), wash buffer (TKR buffer+0.2% w/v OvAlb), binding buffer (wash buffer+0.1 mg/ml Bacitracin), and tracer solution (binding buffer+˜15000 cpm/well ³H-tracer). The assay was performed on ice the day after transfection.

Briefly, maintenance medium was aspirated, and plates were washed ×1 in cold wash buffer before adding 100 μl cold binding buffer and placing at 4° C. to let plates cool. The cold plates were added indicated peptides (ligand) immediately followed by addition of cold tracer solution (˜15000 cpm/well). Plates were immediately moved to 4° C. and incubated for 4 hours. After incubation, binding was stopped by wash ×2 with cold wash buffer before adding 225 μl Ultima Gold XR. Plates were shaken at medium speed for approximately 30 min and left overnight at room temperature before counting the plates in a MicroBeta plate counter (Perkin Elmer).

### Example 4: Determination of Potencies and Efficacies by Measuring Cyclic Adenosine Monophosphate (cAMP)

Materials:

CaCl₂, Tris-HCl, EDTA, HEPES, NaCl, Chloroquine, 3-isobutyl-1-methylxanthine (IBMX), and ovalbumin (albumin from chicken eggs) (Sigma Aldrich). COS-7 monkey kidney cell line was obtained from ATCC. DMEM 1885, FBS, Penicillin/Streptomycin (P/S) and HBSS were from Thermo Scientific/Gibco. Solid white 96 well plates from Corning. Hithunter cAMP assay for Biologics from Discover X. pcDNA3.1(+) containing coding sequences of human and mouse tachykinin receptor 1, 2, 3 mRNA were obtained from Genscript (custom order). Synthesized peptides diluted in saline+0.2% (w/v) ovalbumin.
Human Tacr1: NM_001058.4
Human Tacr2: NM_001057.3
Human Tacr3: NM 001059.2

Methods:

As a secondary measure of agonist-induced G-protein coupled receptor (GPCR) activation cyclin adenosine monophosphate (cAMP) was measured by the Hithunter cAMP-assay from DiscoverX. Assays were carried out using COS-7 cells transiently transfected by calcium phosphate transfection with a vector pcDNA3.1(+) encoding one of the indicated receptors (Genscript). Briefly, DNA mixed with CaCl₂ (2 M) and TE-buffer (10 mM Tris-HCl, 1 mM EDTA, pH 7.5) was dropwise added to 2×HBS (50 mM HEPES, 280 mM NaCl, 1.5 mM NaH₂PO₄, pH 7.2) and incubated for 45 min at room temperature (22±2° C.). The mixture and a final concentration of 100 μM Chloroquine were added to the cells and left to incubate for 5 hours at 37° C. under standard cell culture conditions (10% CO₂) before changing medium to fresh maintenance medium.

The cAMP-assay takes advantage of the tachykinin receptors' ability to induce production of the cAMP second messenger upon agonist (ligand) binding on receptor expressing cells. Production of cAMP stems from receptor coupling to Gs-protein although coupling to Gq-protein (IP₃-production) is considered the primary signalling mechanism by tachykinin receptors.

The assay was performed the day after transfection. Briefly, maintenance medium was aspirated, and plates were washed ×1 in HBSS before adding assay buffer (HBSS+1 mM IBMX), pre-incubated for 30 min at 37° C. followed by 15 min incubation with agonist (ligand) at 37° C. After incubation, plates were subjected to cell lysis and anti-cAMP-antibody incubation as described by manufacturer. Luminescence was measured with EnVision Multimode Plate Reader from Perkin Elmer.

### Example 5: Synthesis and Characterization of Peptides

General Methods

The following relates to methods for synthesising resin bound peptides (SPPS methods, including methods for de-protection of amino acids, methods for cleaving the peptide from the resin, and for its purification), as well as methods for detecting and characterising the resulting peptide (LCMS and UPLC methods).

SPPS Method

The Fmoc-protected amino acid derivatives used were the standard recommended: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(BOC)-OH, Fmoc-Met-OH, Fmoc-Phe-OH, Fmoc-Pro-OH, Fmoc-Ser(tBu)-OH, Fmoc-Thr(tBu)-OH, Fmoc-Trp(BOC)-OH, Fmoc-Tyr(tBu)-OH, Fmoc-Val-OH and Fmoc-Lys(Mtt)-OH supplied from e.g. Anaspec, Bachem, Iris Biotech, or NovabioChem.

The N-terminal amino acid is Boc protected at the alpha amino group (e.g. Boc-Asp(OtBu)-OH for peptides with Asp at the N-terminus).

The introduction of the substituent on the epsilon-nitrogen of a lysine was achieved using a lysine protected with Mtt (Fmoc-Lys(Mtt)-OH). Suitably protected building blocks such as Fmoc-8-amino-3,6-dioxaoctanoic acid, and Fmoc-Glu-OtBu were used for the introduction of the substituent. Introduction of the fatty acid moiety was achieved using building blocks such as octadecanedioic acid mono-tert-butyl-ester.

SPPS was performed on a SymphonyX Solid Phase Peptide Synthesizer from Protein Technologies (Tucson, AZ 85714 U.S.A.) at 100-μmol, 150-μmol, 300 μmol or 450-μmol scale using 4, 6, 12, or 18-fold excess of Fmoc-amino acids (300 mM in DMF with 300 mM Oxyma Pure®) relative to resin loading. Fmoc-PAL AM resin (Novabiochem, loading e.g. 0.61 nmol/g), Rink Amide AM polystyrene resin (Novabiochem, loading e.g. 0.64 mmol/g), or 2-chlorotrityl chlorid resin (loading 1.42 mmol/g) was used as the solid support. Fmoc-deprotection was performed using 20% piperidine in DMF. Coupling was performed using 1:1:1:1 amino acid/(Oxyma Pure®)/DIC/collidine in DMF. DCM (1×1.5 ml) and DMF top washes (6×6 ml) were performed between deprotection and coupling steps. Coupling times were generally 60 minutes (ranging from 60 min to 8 hours). Some amino acids including, but not limited to Fmoc-Arg(Pbf)-OH, and Fmoc-Gly-OH were “double coupled”, meaning that after the first coupling (e.g. 60 min), the resin is drained and more reagents are added (amino acid, Oxyma Pure®, DIC, and collidine), and the mixture allowed to react again (e.g. 60 min). The Mt group was removed by first washing the resin with DCM (1×1 min) followed by suspending the resin in HFIP/DCM/TIS (75/23/2) (1×5 min). The resin was washed with DCM and suspended in HFIP/DCM/TIS (75/23/2) (2×25 min with a DCM wash in between) subsequently washed in sequence with DMF(1×), DCM(4×), DMF(2×), Piperidine/DMF (20:80), DMF(1×), DCM(1×), DMF(6×).

Cleavage from the Resin

After synthesis the resin was washed with DCM, and the peptide was cleaved from the resin by a 2-3-hour treatment with TFA/TIS/water (95/2.5/2.5) followed by precipitation with diethylether. The precipitate was washed with diethylether.

Purification

The crude peptide was dissolved in a suitable solvent mixture (such as e.g. 10/20/70 acetic acid/MeCN/water) and purified by reversed-phase preparative HPLC (Waters Prep) on a column containing C18-silica gel. Elution was performed with an increasing gradient of MeCN in water containing 0.1% TFA or with an increasing gradient of 80:20 MeCN:MQ-water in phosphatebuffer (20 mm Na₂HPO₄, 20 mm NaH₂PO₄, 10% MeCN in MQ at pH 7.2). Relevant fractions were analysed by a combination of UPLC, and LCMS methods, and the appropriate fractions were pooled and freeze dried.

Methods for Detection and Characterization

LCMS Methods

LCMS was performed on a setup consisting of Waters Acquity UPLC system and LCT Premier XE mass spectrometer from Micromass. The analysis was performed at room temperature by injecting an appropriate volume of the sample (preferably 2-10 μl) onto the column (Waters Acquity UPLC BEH, C-18, 1.7 μm, 2.1 mm×50 mm) which was eluted with a gradient of A, B (and D).

Method: LCMS34

Eluents: A: 0.1% Formic acid in MQ-water. B: 0.1% Formic acid in acetonitrile. Gradient: Linear 5%-95% acetonitrile during 4.0 min at 0.4 ml/min. Detection: 214 nm (analogue output from TUV (Tunable UV detector)) MS ionisation mode: API-ES (positive mode). Scan: 100-2000 amu (alternatively 500-2000 amu), step 0.1 amu.

Method: LCMS43

Eluents: A: MQ-water. B: acetonitrile D: 100 mm triethylammonium acetate in water: acetonitrile 1:1 (pH adjusted to 7.8 with Et₃N+AcOH). Gradient: Linear 0%-97.5% acetonitrile+isocratic 2.5% D during 4.0 min at 0.4 ml/min. Detection: 214 nm (analogue output from TUV (Tunable UV detector)) MS ionisation mode: API-ES (negative mode). Scan: 100-2000 amu (alternatively 500-2000 amu), step 0.1 amu.

UPLC Methods

The reverse phase-analysis was performed using a Waters UPLC system fitted with a dual band detector. UV detections at 214 nm were collected using an ACQUITY UPLC BEH, C18, 1.7 um, 2.1 mm×150 mm column. The UPLC system was connected to two eluent reservoirs A and B.

Method: UPLC01:

Column temperature: 40° C. Eluents: A: 99.95% MQ-water, 0.05% TFA, B: 99.95% CH₃CN, 0.05% TFA. The following linear gradient was used: 95% A, 5% B to 40% A, 60% B over 16 minutes at a flow-rate of 0.40 ml/min.

Method: UPLC02:

Column temperature: 40° C. Eluents: A: 99.95% MQ-water, 0.05% TFA, B: 99.95% CH₃CN, 0.05% TFA. The following linear gradient was used: 95% A, 5% B to 5% A, 95% B over 16 minutes at a flow-rate of 0.40 ml/min.

Method: UPLC6

Column temperature: 60° C. Eluents: A: 0.02 m Na₂SO4, 0.002 m Na₂HPO₄, 0.002 m NaHPO₄, B: 70% CH3CN in MQ-water. Step gradient: 10-20% B over 3 minutes, then 20-50% B over 17 minutes, then 50-80% B over 1 minute. Step gradient run-time: 21 minutes at a flow-rate of 0.40 ml/min.

Method: UPLC61:

Column temperature: 60° C. Eluents: A: 0.02 m Na₂SO4, 0.002 m Na₂HPO₄, 0.002 m NaHPO₄, B: 70% CH3CN in MQ-water. Step gradient: 10-20% B over 3 minutes, then 20-80% B over 17 minutes, then 80-90% B over 1 minute. Step gradient run-time: 21 minutes at a flow-rate of 0.40 ml/min.

### Example 6: Structures of Conjugated Moieties

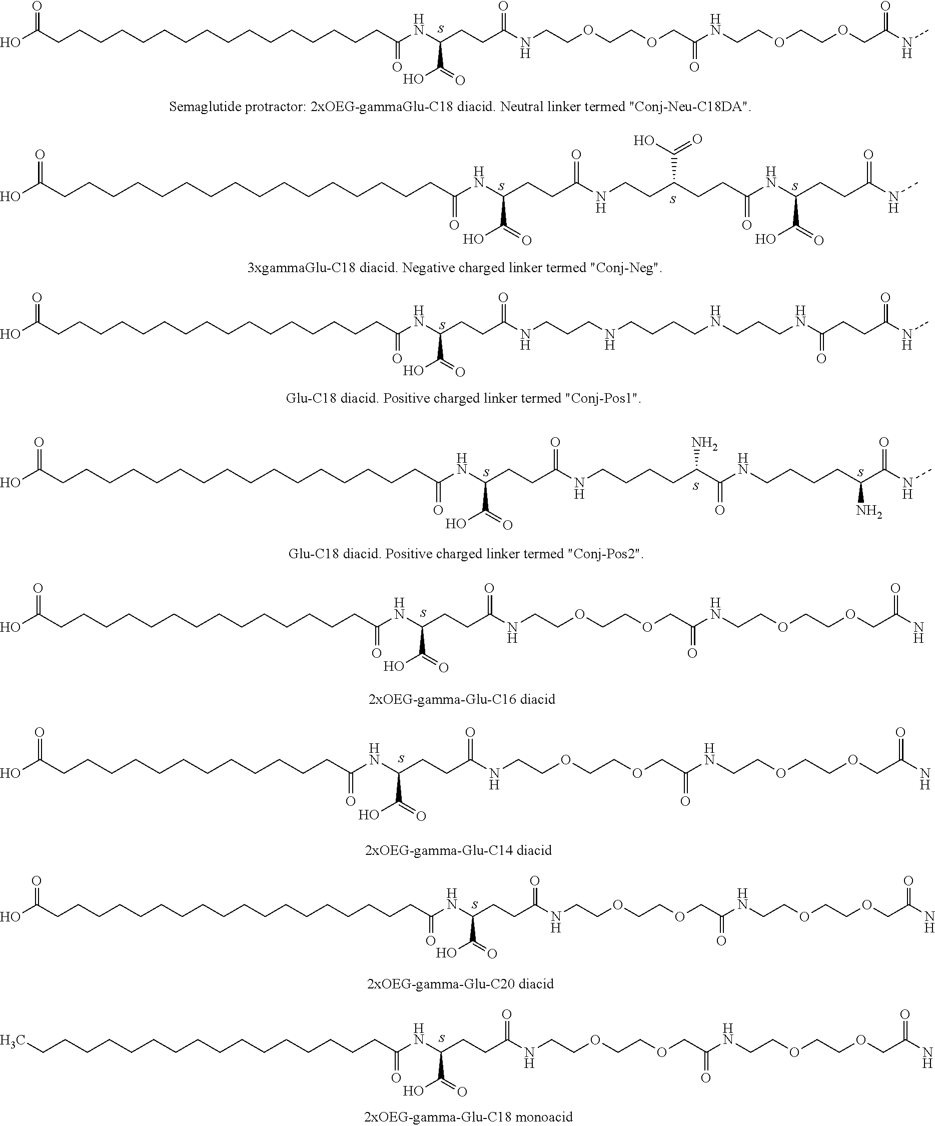

### Example 7: Investigation of Amino Acid Substitutions on NKA(4-10) Analogues for NK2R Activation, Signaling and Selectivity

Position 4 (X₁) Mutations

Selectivity and activation were measured by IP3-assay on human NK1-, NK2- and NK3Rs as described in Example 1. Gs-coupling was investigated by cAMP accumulation as described in Example 4. Binding was measured by competitive ³H-NKA binding as described in Example 3.

Results:

TABLE 7

| | | hNK1R, | | hNK2R, | | hNK3, | | hNK2R, | | |
|---|---|---|---|---|---|---|---|---|---|---|
| | | IP₃ | | IP₃ | | IP₃ | | cAMP | | hNK2R, |
| | | | Effi- | | Effi- | | Effi- | | Effi- | binding |
| | | EC50 | cacy | EC50 | cacy | EC50 | cacy | EC50 | cacy | EC50 |
| ID | Sequence | (nM) | (%) | (nM) | (%) | (nM) | (%) | (nM) | (%) | (nM) |
| 304 | *Asp;Lys;Phe;Val; | 34 | 80 | 2.1 | 90 | 66 | 65 | ND | ND | ND |
| | Gly;NmLeu;Nle;NH2 | | | | | | | | | |
| 335 | *Glu;Lys;Phe;Val; | 36 | 70 | 1.6 | 80 | 113 | 100 | ND | ND | ND |
| | Gly;NmLeu;Nle;NH2 | | | | | | | | | |
| 305 | *Asp;Lys;Tyr;Val; | 520 | 30 | 3.7 | 80 | ~730 | 10 | 0.7 | 50 | 21 |
| | Gly;NmLeu;Nle;NH2 | | | | | | | | | |
| 336 | *Glu;Lys;Tyr;Val; | ~26,000 | 30 | 4.0 | 80 | ~3800 | 10 | — | 0 | 80 |
| | Gly;NmLeu;Nle;NH2 | | | | | | | | | |
| 306 | *Asp;Arg;Phe;Val; | 19 | 80 | 2.0 | 90 | 36 | 65 | ND | ND | ND |
| | Gly;NmLeu;Nle;NH2 | | | | | | | | | |
| 337 | *Glu;Arg;Phe;Val; | 23 | 70 | 4.0 | 80 | 43 | 100 | ND | ND | ND |
| | Gly;NmLeu;Nle;NH2 | | | | | | | | | |

Comparison of selectivity and receptor activation of compounds 304, 335, 305, 336, 306 and 337. The position of the protractor on the amino acid sequence is marked by an asterisk “*”. Unless stated otherwise, “*” is “Conj-Neu-C18DA” the structure of which is illustrated in Example 6. Data are presented as EC50 or efficacy calculated by nonlinear regression using Sigmoidal, 4PL, X is log(concentration) equation in Graphpad Prism 8.
ND: not determined.

Summary
Asp4-to-Glu4 substitution does not change hNK2R IP3 activation on NKA(4-10) analogues (compounds: 304-337).
Asp4-to-Glu4 substitution does not change hNK2R selectivity on Tyr-analogue, but reduces binding affinity (compounds: 305 and 336).
Glu4 Tyr-analogue does not activate Gs (compound 336).

Position 5 (X₂) Mutations

Selectivity and activation were measured by IP3-assay on human NK1-, NK2- and NK3Rs as described in Example 1.

TABLE 8

| | | hNK1R, IP₃ | | hNK2R, IP₃ | | hNK3, IP₃ | |
|---|---|---|---|---|---|---|---|
| | | EC50 | Efficacy | EC50 | Efficacy | EC50 | Efficacy |
| ID | Sequence | (nM) | (%) | (nM) | (%) | (nM) | (%) |
| 304 | *Asp;Lys;Phe;Val;Gly;NmLeu;Nle;NH2 | 34 | 80 | 2.1 | 90 | 66 | 65 |
| 306 | *Asp;Arg;Phe;Val;Gly;NmLeu;Nle;NH2 | 19 | 80 | 2.0 | 90 | 36 | 65 |
| 335 | *Glu;Lys;Phe;Val;Gly;NmLeu;Nle;NH2 | 36 | 70 | 1.6 | 80 | 113 | 100 |
| 336 | *Glu;Lys;Tyr;Val;Gly;NmLeu;Nle;NH2 | ~27,000 | 30 | 4.0 | 80 | ~3800 | 10 |
| 337 | *Glu;Arg;Phe;Val;Gly;NmLeu;Nle;NH2 | 23 | 70 | 1.3 | 80 | 43 | 100 |
| 357 | *Glu;Arg;Tyr;Val;Gly;NmLeu;Nle;NH2 | — | 50 | 6.5 | 90 | — | 10 |

Results obtained by investigating compounds 304 vs 306 (Lys5 → Arg5), 337 vs 335, 336 vs 357. The position of the protractor on the amino acid sequence is marked by an asterisk “*”. Unless stated otherwise, “*” is “Conj-Neu-C18DA” the structure of which is illustrated in Example 6. Data are presented as EC50 or efficacy calculated by nonlinear regression using Sigmoidal, 4PL, X is log(concentration) equation in Graphpad Prism 8.
ND: not determined.

Summary
Position 5 (X₂) was tested using arginine substitution. In general, arginine substitution did not affect NK2R activation, selectivity or bias (compounds:304-357).
On Glu4_Tyr6 analogues, Arg5 substitution could increase NK2- and NK1R efficacy, without affecting Gq bias (compounds: 336, 337 and 357).
Arg5 substitution increases NK3R potency on NKA(4-10)Glu4 analogue (compounds: 335 and 337).

Position 6 (X₃) Mutations

Selectivity and activation were measured by IP3-assay on human NK1-, NK2- and NK3Rs as described in Example 1. Gs-coupling was investigated by cAMP accumulation as described in Example 4. Binding was measured by competitive 3H-NKA binding as described in Example 3.

TABLE 9

| | | | | | | | | hNK2R, | | hNK2R, |
|---|---|---|---|---|---|---|---|---|---|---|
| | | hNK1R, IP₃ | | hNK2R, IP₃ | | hNK3, IP₃ | | cAMP | | binding |
| | | EC50 | Efficacy | EC50 | Efficacy | EC50 | Efficacy | EC50 | Efficacy | EC50 |
| ID | Sequence | (nM) | (%) | (nM) | (%) | (nM) | (%) | (nM) | (%) | (nM) |
| 304 | *Asp;Lys;Phe;Val; | 40 | 80 | 2.1 | 90 | 66 | 60 | 2.3 | 100 | 25 |
| | Gly;NmLeu;Nle;NH2 | | | | | | | | | |
| 305 | *Asp;Lys;Tyr;Val; | 520 | 30 | 3.7 | 80 | ~720 | 0 | 0.7 | 50 | 210 |
| | Gly;NmLeu;Nle;NH2 | | | | | | | | | |
| 361 | *Asp;Lys;3-OH-Phe; | 200 | 70 | 2.3 | 100 | ~47,000 | 60 | ND | ND | ND |
| | Val;Gly;NmLeu;Nle; | | | | | | | | | |
| | NH2 | | | | | | | | | |
| 362 | *Asp;Lys;Pro;Val; | — | 0 | — | 0 | — | 0 | ND | ND | ND |
| | Gly;NmLeu;Nle;NH2 | | | | | | | | | |
| 363 | *Asp;Lys;Val;Tyr; | — | 0 | — | 0 | — | 0 | ND | ND | ND |
| | Gly;NmLeu;Nle;NH2 | | | | | | | | | |
| 330 | *Asp;Lys;4-I-Phe; | ~170 | 40 | — | 0 | — | 0 | ND | ND | ND |
| | Val;Gly;NmLeu;Nle; | | | | | | | | | |
| | NH2 | | | | | | | | | |
| 356 | *Asp;Lys;dPhe;Val; | — | 0 | — | 0 | — | 0 | ND | ND | ND |
| | Gly;NmLeu;Nle;NH2 | | | | | | | | | |

Comparison of selectivity and receptor activation of compounds 304, 305, 361, 362, 363, 330, and 356. NmLeu is L-N-methylleucine. The position of the protractor on the amino acid sequence is marked by an asterisk “*”. Unless stated otherwise, “*” is “Conj-Neu-C18DA” the structure of which is illustrated in Example 6. Data are presented as EC50 or efficacy calculated by nonlinear regression using Sigmoidal, 4PL, X is log(concentration) equation in Graphpad Prism 8.
ND: not determined.

Summary
Phe6 to Tyr6 substitution on NKA(4-10) analogues promotes NK2R-selectivity with a slight loss of efficacy, receptor binding and Gs-coupling efficacy (compounds: 304 and 305).
Changing position of the hydroxyl group on the phenyl group of tyrosine, from position 4 to position 3 on the phenyl of Phe6, results in decreased NK2R selectivity and restores NK1R efficacy to Phe-analogue levels (compounds: 304, 305 and 361).
Introduction of proline or switch of Ty6 and Val7 causes a complete loss of receptor activation on NK1-3Rs (compounds: 362 and 363).
I-4-Phe and dPhe substitution completely destroys NK2R activation (compounds: 330 and 356).

Position 7 (X₄) Mutations

Selectivity and activation were measured by IP3-assay on human NK1-, NK2- and NK3Rs as described in Example 1. Gs-coupling was investigated by cAMP accumulation as described in Example 4. Binding was measured by competitive 3H-NKA binding as described in Example 3.

Results

TABLE 10

| | | | | | | | | hNK2R, | | hNK2R, |
|---|---|---|---|---|---|---|---|---|---|---|
| | | hNK1R, IP₃ | | hNK2R, IP₃ | | hNK3, IP₃ | | cAMP | | binding |
| | | EC50 | Efficacy | EC50 | Efficacy | EC50 | Efficacy | EC50 | Efficacy | EC50 |
| ID | Sequence | (nM) | (%) | (nM) | (%) | (nM) | (%) | (nM) | (%) | (nM) |
| 314 | *Glu;Lys;Tyr;Arg; | — | 0 | ~3100 | 45 | — | 0 | | | |
| | Gly;NmLeu;Nle;NH2 | | | | | | | | | |
| 315 | *Glu;Lys;Phe;Arg; | — | 0 | 47 | 60 | — | 0 | | | |
| | Gly;NmLeu;Nle;NH2 | | | | | | | | | |
| 335 | *Glu;Lys;Phe;Val; | 36 | 72 | 1.6 | 80 | 113 | 100 | | | |
| | Gly;NmLeu;Nle;NH2 | | | | | | | | | |
| 305 | *Asp;Lys;Tyr;Val; | 520 | 30 | 3.7 | 100 | ~720 | 0 | 0.7 | 50 | 210 |
| | Gly;NmLeu;Nle;NH2 | | | | | | | | | |
| 348 | *Asp;Lys;Tyr; | 191 | 50 | 3.5 | 100 | — | 0 | — | — | 1400 |
| | Ile(S);Gly;NmLeu; | | | | | | | | | |
| | Nle;NH2 | | | | | | | | | |
| 351 | *Asp;Lys;Tyr; | 950 | 50 | 14 | 100 | — | 0 | — | — | — |
| | Ile(R);Gly;NmLeu; | | | | | | | | | |
| | Nle;NH2 | | | | | | | | | |
| 304 | *Asp;Lys;Phe;Val; | 34 | 80 | 2.1 | 100 | 66 | 60 | ND | ND | ND |
| | Gly;NmLeu;Nle;NH2 | | | | | | | | | |
| 387 | *Asp;Lys;Phe;Thr; | 5.5 | 100 | 3.3 | 100 | 34 | 100 | ND | ND | ND |
| | Gly;NmLeu;Nle;NH2 | | | | | | | | | |
| 336 | *Glu;Lys;Tyr;Val; | ~26000 | 25 | 4.0 | 75 | — | 0 | | | |
| | Gly;NmLeu;Nle;NH2 | | | | | | | | | |
| 397 | *Glu;Lys;Phe;Thr; | — | 0 | 28 | 75 | — | 0 | | | |
| | Gly;NmLeu;Nle;NH2 | | | | | | | | | |
| 344 | *Asp;Lys;Tyr;Val; | ~160 | 30 | 2.2 | 90 | ~24000 | 30 | 28 | 96 | 94 |
| | Gly;NmLeu;Metox; | | | | | | | | | |
| | NH2 | | | | | | | | | |
| 366 | *Asp;Lys;Tyr;Thr; | — | 0 | 12 | 100 | — | 0 | ~120 | 80 | ~104 |
| | Gly;NmLeu;Metox; | | | | | | | | | |
| | NH2 | | | | | | | | | |
| 381 | *Glu;Lys;Tyr;Thr; | — | 0 | 9.3 | 85 | — | 0 | | | |
| | Gly;NmLeu;Metox; | | | | | | | | | |
| | NH2 | | | | | | | | | |
| 382 | *Glu;Lys;Tyr;Val; | ~3800 | 30 | 3.7 | 95 | — | 20 | | | |
| | Gly;NmLeu;Metox; | | | | | | | | | |
| | NH2 | | | | | | | | | |
| 383 | *Asp;Lys;Phe;Thr; | 290 | 50 | 1.6 | 95 | 313 | 60 | ~11 | 89 | 11 |
| | Gly;NmLeu;Metox; | | | | | | | | | |
| | NH2 | | | | | | | | | |
| 384 | *Asp;Lys;Phe;Val; | 16 | 80 | 0.9 | 95 | 10 | 85 | | | |
| | Gly;NmLeu;Metox; | | | | | | | | | |
| | NH2 | | | | | | | | | |
| 386 | *Asp;Lys;Tyr;Ser; | — | 0 | 29 | 63 | — | 0 | | | |
| | Gly;NmLeu;Nle;NH2 | | | | | | | | | |

Comparison of selectivity and receptor activation of compounds 314, 315, 335, 305, 348, 351, 304, 387, 336, 397, 344, 366, 381, 382, 383, 384, and 386. The position of the protractor on the amino acid sequence is marked by an asterisk “*”. Unless stated otherwise, “*” is “Conj-Neu-C18DA” the structure of which is illustrated in Example 6. Data are presented as EC50 or efficacy calculated by nonlinear regression using Sigmoidal, 4PL, X is log(concentration) equation in Graphpad Prism 8.
ND: not determined.

Summary
Arginine substitution on position 7 causes loss of receptor activation properties on NKA(4-10) analogues (compounds: 314, 315, 335 and 336).
Isoleucine (S-isoform) substitution on position 7 is possible and does not change potency, efficacy and selectivity of Tyr6 NKA(4-10) analogues. However, Isoleucine (R-isoform) decreases NK2R potency. Both Isoleucine substitutions reduce Gs signalling and receptor binding capacity (compounds: 348 and 351).
Threonine substitution on NKA(4-10) analogues reduces NK2R activity, but can work as a selectivity driver in Phe6-NKA(4-10) analogues. Thr7 promotes receptor binding and sustains Gs activation similar to NKA (compounds: 304, 387, 305, 397, 344, 366, 381, 382, 383, 384).
Serine substitution on position 7 reduces NK2R potency and efficacy without affecting selectivity (compounds: 305 and 386).

Position 8 (X₅) Mutations

Selectivity and activation were measured by IP3-assay on human NK1-, NK2- and NK3Rs as described in Example 1. Binding was measured by competitive 3H-NKA binding as described in Example 3.

TABLE 11

| | | | | | | | | hNK2R, |
|---|---|---|---|---|---|---|---|---|
| | | hNK1R, IP₃ | | hNK2R, IP₃ | | hNK₃, IP3 | | binding |
| | | EC50 | Efficacy | EC50 | Efficacy | EC50 | Efficacy | EC50 |
| ID | Sequence | (nM) | (%) | (nM) | (%) | (nM) | (%) | (nM) |
| 310 | *Lys;Thr;Asp;Ser;Phe;Val;bAla;NmLeu;Nle;NH2 | — | 0 | 45 | 65 | — | 80 | |
| 312 | *Lys;Thr;Asp;Ser;Phe;Val;Gly;NmLeu;Nle;NH2 | ~178 | 44 | 25 | 65 | 513 | 80 | |
| 322 | *Lys;Thr;Asp;Ser;Phe;Val;bAla;Leu;Nle;NH2 | — | 0 | ~130 | 53 | ~160 | 80 | |
| 308 | *Lys;Thr;Asp;Ser;Phe;Val;Gly;Leu;Nle;NH2 | ~164 | 60 | 96 | 65 | 1000 | 93 | |
| 389 | *Asp;Lys;Phe;Val;Gly;Leu;Nle;NH2 | 16 | 108 | 2.8 | 83 | 35 | 100 | |
| 373 | *Asp;Lys;Phe;Val;Aib;Leu;Nle;NH2 | ~136 | 66 | 4.3 | 89 | — | 0 | |
| 402 | *Asp;Lys;Tyr;Val;dSer;Leu;Nle;NH2 | — | 0 | 15 | 77 | — | 0 | 121 |
| 392 | *Asp;Lys;Tyr;Val;Gly;Leu;Nle;NH2 | 205 | 52 | 5.4 | 72 | — | 0 | 25 |
| 385 | *Asp;Lys;Phe;Val;dSer;Leu;Nle;NH2 | — | 0 | 4.5 | 46 | — | 0 | |
| 396 | *Asp;Lys;Tyr;Val;dSer;Leu;Metox;NH2 | — | 0 | 20 | 92 | — | 0 | 316 |
| 393 | *Asp;Lys;Tyr;Val;Gly;Leu;Metox;NH2 | 342 | 52 | 10 | 100 | — | 36 | 40 |

Comparison of selectivity and receptor activation of compounds 310, 312, 322, 308, 389, 373, 402, 392, 385, 396, and 393. The position of the protractor on the amino acid sequence is marked by an asterisk “*”. Unless stated otherwise, “*” is “Conj-Neu-C18DA” the structure of which is illustrated in Example 6. Data are presented as EC50 or efficacy calculated by nonlinear regression using Sigmoidal, 4PL, X is log(concentration) equation in Graphpad Prism 8.
ND: not determined.

Summary
Beta-alanine substitution on NKA (2-10) promotes NK2- and NK3R selectivity, but decreases NK2R potency and efficacy (compounds: 310, 312, 322 and 308).
Aib8 substitution promotes selectivity in Phe6-NKA(4-10) analogues (compounds: 389 and 373).
Position 8 d-Ser substitution promotes selectivity in NKA(4-10) analogues with endogenous NKA(4-10) backbone as well as Phe6- and Tyr6-NKA(4-10) analogues, but reduces NK2R affinity (compounds: 389, 402, 392, 385, 396, 393).

Position 9 (X₆) Mutations

Selectivity and activation were measured by IP₃-assay on human NK1-, NK2- and NK3Rs as described in Example 1.

TABLE 12

| | | hNK1R, IP₃ | | hNK2R, IP₃ | | hNK3, IP₃ | |
|---|---|---|---|---|---|---|---|
| | | EC50 | Efficacy | EC50 | Efficacy | EC50 | Efficacy |
| ID | Sequence | (nM) | (%) | (nM) | (%) | (nM) | (%) |
| 304 | *Asp;Lys;Phe;Val;Gly;NmLeu;Nle;NH2 | 34 | 80 | 2.1 | 100 | 65 | 100 |
| 389 | *Asp;Lys;Phe;Val;Gly;Leu;Nle;NH2 | 15 | 100 | 2.9 | 80 | 35 | 60 |
| 305 | *Asp;Lys;Tyr;Val;Gly;NmLeu;Nle;NH2 | 520 | 30 | 3.7 | 80 | ~730 | 0 |
| 392 | *Asp;Lys;Tyr;Val;Gly;Leu;Nle;NH2 | 200 | 30 | 5.4 | 70 | — | 0 |
| 344 | *Asp;Lys;Tyr;Val;Gly;NmLeu;Metox;NH2 | — | 50 | 3.1 | 100 | — | 30 |
| 393 | *Asp;Lys;Tyr;Val;Gly;Leu;Metox;NH2 | 3400 | 50 | 10 | 100 | — | 30 |

Comparison of selectivity and receptor activation of compounds 304, 389, 305, 392, 344, and 393. The position of the protractor on the amino acid sequence is marked by an asterisk “*”. Unless stated otherwise, “*” is “Conj-Neu-C18DA” the structure of which is illustrated in Example 6. Data are presented as EC50 or efficacy calculated by nonlinear regression using Sigmoidal, 4PL, X is log(concentration) equation in Graphpad Prism 8.
ND: not determined.

Summary
N-methyl-leucine induces a modest potency increase on hNK2R on NKA(4-10) analogues (compounds: 304-393).
N-Me-Leu induces NK2R selectivity in in NKA(4-10) analogues (compounds: 304-393).

Position 10 (X₇) Mutations

Selectivity and activation were measured by IP3-assay on human NK1-, NK2- and NK3Rs as described in Example 1. Gs-coupling was investigated by cAMP accumulation as described in Example 4. Binding was measured by competitive ³H-NKA binding as described in Example 3.

TABLE 13

| | | | | | | | | hNK2R, | | hNK2R, |
|---|---|---|---|---|---|---|---|---|---|---|
| | | hNK1R, IP₃ | | hNK2R, IP₃ | | hNK3, IP₃ | | cAMP | | binding |
| | | EC50 | Efficacy | EC50 | Efficacy | EC50 | Efficacy | EC50 | Efficacy | EC50 |
| ID | Sequence | (nM) | (%) | (nM) | (%) | (nM) | (%) | (nM) | (%) | (nM) |
| 316 | *Asp;Lys;Tyr;Val; | 20 | 100 | 12 | 120 | ~160 | 50 | 20 | 100 | 41 |
| | Gly;NmLeu;Met;NH2 | | | | | | | | | |
| 305 | *Asp;Lys;Tyr;Val; | — | ~30 | 4 | 100 | — | 40 | ~0.8 | 70 | ~100 |
| | Gly;NmLeu;Nle;NH2 | | | | | | | | | |
| 344 | *Asp;Lys;Tyr;Val; | — | ~30 | 5 | 110 | ~170 | 0 | 28 | 100 | 90 |
| | Gly;NmLeu;Metox; | | | | | | | | | |
| | NH2 | | | | | | | | | |
| 369 | *Asp;Lys;Tyr;Val; | ~250 | 40 | 1.7 | 90 | ~190 | 25 | 54 | 80 | 76 |
| | Gly;NmLeu;4-F-Phe; | | | | | | | | | |
| | NH2 | | | | | | | | | |
| 353 | *Asp;Lys;Tyr;Val; | — | 20 | 2.5 | 90 | — | 0 | ~1.3 | 50 | 27 |
| | Gly;NmLeu;Phe;NH2 | | | | | | | | | |
| 313 | *Asp:Lys:Tyr:Val: | — | 20 | 23 | 70 | — | 0 | ND | ND | ND |
| | Gly:NmLeu:cHexAla: | | | | | | | | | |
| | NH2 | | | | | | | | | |
| 370 | *Asp:Lys:Phe:Val: | — | 20 | 10 | 90 | — | 0 | ND | ND | ND |
| | Gly:NmLeu:4-MeOPhe: | | | | | | | | | |
| | NH2 | | | | | | | | | |
| 395 | *Asp;Lys;Tyr;Val; | — | 0 | 9 | 68 | — | 0 | — | 0 | ~104 |
| | Aib;Leu;Nle;NH2 | | | | | | | | | |
| 394 | *Asp;Lys;Tyr;Val; | ~940 | 35 | 5 | 90 | — | 0 | 306 | 100 | ~108 |
| | Aib;Leu;Metox;NH2 | | | | | | | | | |

Comparison of selectivity and receptor activation of compounds 316, 305, 344, 369, 353, 313, 370, 395, and 394. cHexAla is L-cyclohexylalanine. 4-MeOPhe is L-4-Methoxyphenylalanine. The position of the protractor on the amino acid sequence is marked by an asterisk “*”. Unless stated otherwise, “*” is “Conj-Neu-C18DA” the structure of which is illustrated in Example 6. Data are presented as EC50 or efficacy calculated by nonlinear regression using Sigmoidal, 4PL, X is log(concentration) equation in Graphpad Prism 8.
ND: not determined.

Summary
Methionine at position 10 improves NK2R activation and binding compared to norleucine and methoxinine. However, methoxinine and norleucin provides better selectivity with methoxinine-analogues being more potent compared to norleucine-analogues (compounds: 316, 305, 344, 394 and 395).
Substitution of methionine with 4F-Phe is possible while maintaining selectivity, potency, signalling and binding (compounds: 313, 353, 369 and 370).

### Example 8: Materials and Methods for In Vivo Studies

Materials:

NaH2PO4·H2O, Na2HPO4·2H2O, propylene glycol, maintenance diet for rats and mice (regular chow, #1320, Altromin), C57BL/6NRj mice (Janvier Labs), high fat diet (HFD) with 60% energy from fat (#D12492, Research Diets Inc.), peptide analogues (Novo Nordisk), d-glucose (Sigma), insulin (Novo Nordisk), sterile saline solution (Apoteket), Tween-80 (Sigma), Loperamide (Sigma), glucometer and glucose strips (Bayer), and Promethion System (Sable Systems International) for assessment of metabolic and behavioral information.

Time of flight liquid chromatography mass spectrometry (TF-LC-MS): ethanol, methanol, acetonitrile, formic acid, milli-q-water, TurboFlow Cyclone column 0.5×50 mm, Aeris Peptide XB-C18 2.1×50 mm (3.6 μm), Thermo TSQ Altis triple quadrupole Mass spectrometer.

Metabolite identification liquid chromatography mass spectrometry (MetID-LC-MS): methanol, acetonitrile, formic acid, milli-q-water, Water Acquity UPLC Protein BEH C4 2.1×50 mm 300 Å (1.7 μm), Bruker MaXis QTOF.

In vivo buffer for peptide analogues: 8 mM phosphate and 240 mM propylene glycol, pH 8.2.

In vivo buffer for Loperamide: Saline supplemented with 1% (v/v) Tween-80.

Methods:

Animals were housed with access to maintenance diet from weaning till around 6-10 weeks of age. At any time, except from fasting, mice had ad libitum access to food and water with a 12-hour light-dark cycle and 22-24 degree Celsius temperature. All animal experiments were performed according to Danish Animal Inspectorate regulations. For studies using diet-induced obese mice, mice were fed a HFD for at least 20 weeks prior to experimentation. Specifically, for mice undergoing glucose and insulin tolerance tests, mice above 45 g were selected.

Indirect calorimetry was used to evaluate the ability of individual peptide analogues to dose-dependently increase energy expenditure (EE) we used metabolic cages and indirect calorimetry measured by the Promethion system. To this end, oxygen consumption was used as a surrogate measure for EE. Substrate preference (fat or carbohydrate) was evaluated using the respiratory exchange ratio (RER). In parallel, behavioral information such as walking distance and water and food intake were recorded.

Prior to experimentation, DIO mice were transferred to habituation cages for at least 10 days (5 days outside and at least 5 days in the Sable Systems gas analyzer module) to acclimatize. For all in vivo compound tests for EE evaluation, mice received subcutaneous injections between 2 and 4 pm.

Pharmacokinetics: To evaluate the in vivo half-life of individual peptide analogues we used wild type lean mice at around 10 weeks of age injected once with a peptide analogue. For each sample time point 3-4 mice were used and blood was drawn from the submandibular vein at indicated time points following injection. Prior to injection mice were given ad libitum access to standard chow diet. Mice were subcutaneously injected with 0.5 mg/kg peptide analogue in a volume of 2 ml/kg.

The amount of peptide analogue and metabolite(s) present in blood samples were measured by TF-LC-MS and MetID-LC-MS, respectively.

TF-LC-MS:

Sample preparation: One volume of plasma is precipitated with three volumes of ethanol (with internal standard). The mixture is centrifuged at 13000 g for 20 min. One volume of supernatant is diluted with two volumes of Milli-Q water (1% formic acid).

Calibration curve: peptide analogue was spiked into blank mouse plasma. Range: 0.5 to 2000 nM (linear 1/x2).

Chromatography, Mobile phase: Mobile phase A: 5% (50/50 methanol/acetonitrile)+95% Milli-Q+1% formic acid. Mobile phase B: 5% Milli-Q+95% (50/50 methanol/acetonitrile)+1% formic acid.

Columns: TurboFlow Cyclone 0.5×50 mm and Aeris Peptide XB-C18 2.1×50 mm (3.6 μm)

Mass spectrometry: Thermo TSQ Altis triple quadrupole, Positive electrospray ionisation mode, MRM-mode.

MetID-LC-MS:

Sample preparation: One volume of plasma is precipitated with three volumes of methanol. The mixture is centrifuged at 13000 g for 20 min. One volume of supernatant is diluted with two volumes of Milli-Q water (1% formic acid)

Calibration curve: peptide analogue was spiked into blank mouse plasma. Range: 20, 200 and 2000 nM (linear 1/x2)

Chromatography: Mobile phase A: 0.1% formic acid in Milli-Q water. Mobile phase B: 0.1% formic acid in acetonitrile.

Column: Water Acquity UPLC Protein BEH C4 2.1×50 mm 300 Å (1.7 μm)

Mass spectrometry: Bruker MaXis QTOF, Positive electrospray ionisation mode

Full scan (m/z from 300 to 1800) and MS/MS.

Energy expenditure screening: After habituation, mice were subcutaneously injected with 0.5 mg/kg peptide analogue in a volume of 2 ml/kg. Mice were injected q.a.d. (quaque altera die) and received a total of two injections. EE was evaluated and increase over vehicle was calculated as percent of mean oxygen consumption over a 30-hour period after injection. Prior to injection peptide analogues were dissolved to 0.25 mg/ml in in vivo buffer.

Energy expenditure and weight loss pharmacodynamics: To evaluate the ability of individual peptide analogues to dose-dependently increase EE by indirect calorimetry in DIO mice. Body weights were monitored from beginning of habituation until end of experiment. After habituation, mice were treated with daily injections of NKA(4-10) analogues at four different doses or vehicle by daily subcutaneous injections for nine days.

EE, body weight, food intake, water intake and walking distance were observed for all 9 days. EE increase over vehicle was calculated as percent of mean oxygen consumption over a 30-hour period after injection. Prior to injection peptide analogues were dissolved and diluted in in vivo buffer. In order to take into account, the different half-lives for the compounds tested, the doses for each compound were calculated based on the given compound's individual pk-profiles. Target AUCs for each compound were calculated relative to compound 305 as seen below in order to reach same AUC of tested compounds.
305 pK dose: 330 nmol/kg
305 Cmax: 3453 nM
305 AUC: 28771

Insulin tolerance test: The effect on insulin tolerance was determined using intraperitoneal insulin tolerance test (ipITT) 24 hours after single subcutaneous injection of NK2R-selective analogue in DIO mice. At day of experimentation, mice were fasted two hours prior to receiving 1.5 U/kg insulin diluted in saline solution (0.2 mL/kg) by intraperitoneal injection. Change in glucose was monitored using glucometer.

Glucose tolerance test: The effect on glucose tolerance was determined using intraperitoneal glucose tolerance test (ipGTT) 24 hours after single subcutaneous injection of NK2R-selective analogue in DIO mice. At day of experimentation, mice were fasted four hours prior to receiving 1 g/kg glucose diluted in saline solution (0.1 mL/kg) by intraperitoneal injection. Change in glucose was monitored using glucometer.

Dysfunctional voiding test: To investigate the effect on dysfunctional voiding, constipation was induced in lean wild-type, male and female, mice using Loperamide (5 mg/kg). 30 minutes after gavage, mice were subcutaneously dosed with either vehicle, 130, 260 and for males also 325 nmol/kg of EB344. 6 hours post Loperamide gavage, mice were removed from the cage and number of feces pellets were counted.

### Example 9: Investigation of Positioning and Composition of “Conjugated Moiety” on NKA and NKA(4-10) Analogues for NK2R Activation, Signalling and Selectivity

Position and composition of “conjugated moiety” i.e. “protractor” was investigated on endogenous NKA and NK2R-selective NKA(4-10) analogues.

Protractor position was addressed in the N-terminal region of NKA(4-10) analogues.

Charge of linker between peptide backbone and fatty acid was investigated.

Fatty acid species was investigated.

Selectivity and activation were measured by IP3-assay on human NK1-, NK2- and NK3Rs as described in Example 1. Gs-coupling was investigated by cAMP accumulation as described in Example 4. Binding was measured by competitive 3H-NKA binding as described in Example 3. Albumin binding was determined by receptor activation, using IP3 assay, in presence or absence of 1% human serum albumin as described in example 2. Energy expenditure was measured in diet-induced obese mice housed in metabolic cages as described in example 8. Pharmacokinetics and exposure were measured in plasma from lean mice treated with compound as described in example 8.

TABLE 14

| | | | hNK1R, IP₃ | | hNK2R, IP₃ | | hNK3, IP₃ | |
|---|---|---|---|---|---|---|---|---|
| | | | EC50 | Efficacy | EC50 | Efficacy | EC50 | Efficacy |
| ID | Sequence | Protractor | (nM) | (%) | (nM) | (%) | (nM) | (%) |
| 301 | **Asp;Lys*;Phe;Val; | * is Conj-Neu- | ~1500 | 64 | 38 | 63 | 160 | 85 |
| | Gly;NmLeu;Met;NH2 | C18DA & | | | | | | |
| | | ** is acetyl | | | | | | |
| 302 | Asp;Lys*;Phe;Val; | * is Conj-Neu- | — | 0 | | 52 | ~2200 | 65 |
| | Gly;NmLeu;Met;NH2 | C18DA | | | | | | |
| 304 | *Asp;Lys;Phe;Val; | * is Conj-Neu- | 12-250 | 80-85 | 1-11 | 80-90 | 60-~7000 | 85-100 |
| | Gly;NmLeu;Nle;NH2 | C18DA | | | | | | |
| 305 | *Asp;Lys;Tyr;Val; | * is Conj-Neu- | 450-3500 | 17-50 | 2.2-13 | 170-75 | | 0 |
| | Gly;NmLeu;Nle;NH2 | C18DA | | | | | | |
| 307 | Asp;Lys*;Phe;Val; | * is Conj-Neu- | — | 64 | 24 | 80 | ~12000 | 85 |
| | Gly;NmLeu;Nle;NH2 | C18DA | | | | | | |
| 318 | *Asp;Lys;Tyr;Val; | * is Conj- | ~780 | 17 | 44 | 85 | | 0 |
| | Gly;NmLeu;Nle;NH2 | Pos2-C18DA | | | | | | |
| 319 | *Asp;Lys;Tyr;Val; | * is Conj-Neg- | - | 0 | ~220 | 40 | | 0 |
| | Gly;NmLeu;Nle;NH2 | C18DA | | | | | | |
| 321 | Asp;Lys;Tyr;Val; | * is Conj- | ~170 | 45 | 90 | 70 | | 0 |
| | Gly;NmLeu;Nle;NH2 | Pos1-C18DA | | | | | | |
| 334 | Asp;Lys*;Tyr;Val; | * is Conj-Neg- | — | 0 | — | 0 | ~26000 | 25 |
| | Gly;NmLeu;Nle-NH2 | C18DA | | | | | | |
| 344 | Asp;Lys;Tyr;Val; | * is Conj-Neu- | — | 30 | 3.2 | 100 | | 30 |
| | Gly;NmLeu;Metox;NH2 | C18DA | | | | | | |
| 367 | *Asp;Lys;Tyr;Val; | * is Conj-Neu- | ~31000 | 20 | 5.6 | 75 | | 0 |
| | Gly;NmLeu;Nle;NH2 | C16DA | | | | | | |
| 368 | *Asp;Lys;Tyr;Val; | * is Conj-Neu- | — | 0 | 19 | 70 | | 0 |
| | Gly;NmLeu;Nle;NH2 | C14DA | | | | | | |
| 374 | Asp;Lys;Tyr;Val; | No protractor | 200 | 50 | 5.8 | 90 | | 0 |
| | Gly;NmLeu;Nle;NH2 | | | | | | | |
| 375 | Asp;Lys;Phe;Val; | No protractor | 12 | 110 | 0.5 | 100 | ~50000 | 35 |
| | Gly;NmLeu;Nle;NH2 | | | | | | | |
| 380 | *Asp;Lys;Tyr;Val; | * is Conj-Neu- | 58 | 20 | 1.5 | 90 | ~160 | 30 |
| | Gly;NmLeu;Nle;NH2 | C20DA | | | | | | |
| 390 | *Asp;Lys;Tyr;Val; | * is Conj-Neu- | 3.9 | 88 | 1 | 100 | 14 | 100 |
| | Gly;NmLeu;Nle;NH2 | C18MA | | | | | | |
| 391 | *Asp;Lys;Tyr;Val; | * is Conj-Neu- | 3.4 | 100 | 0.9 | 90 | 4 | 100 |
| | Gly;NmLeu;Metox;NH2 | C18MA | | | | | | |

Comparison of protractor composition on receptor activity and selectivity on NK2R-selective NKA(4-10) analogues. The position of the protractors on the amino acid sequence is marked by an asterisk “*”, and the structures of the protractors/conjugated moieties can be found in Example 6.
Conj: conjugate;
Neu: neutral charge;
Pos: positive charge;
Neg: negative charge;
MA: monoacid;
DA: diacid.
Cxx refers to the length of carbon atoms in lipid, i.e. C18 contains eighteen carbon atoms.

TABLE 15

| | | | | | | | In vivo efficacy | | |
|---|---|---|---|---|---|---|---|---|---|
| | | | | | | | | | Energy |
| | | | hNK2R, Albumin binding | | | | | | expenditure |
| | | | | | EC50 | Efficacy | | | (percent |
| | | | | | (nM)- | (%)- | | | of vehicle; |
| | | | | | 1% | 1% | Plasma | | based on |
| | | | EC50 | Efficacy | human | human | exposure | Half- | 30 h |
| | | | (nM)- | (%)- | serum | serum | [2 h/26 h] | life | average |
| ID | Sequence | Protractor | ovalbumin | ovalbumin | albumin | albumin | (nM) | (h) | vO2) |
| 305 | *Asp;Lys;Tyr;Val; | * is Conj- | 3.7 | 83 | 260 | 45-51 | 5364/183 | 5.5 | 9 |
| | Gly;NmLeu;Nle;NH2 | Neu-C18DA | | | | | | | |
| 318 | *Asp;Lys;Tyr;Val; | * is Conj- | 6.7 | 77 | 1100 | 42 | 237/BLLQ | — | 0 |
| | Gly;NmLeu;Nle;NH2 | Pos2-C18DA | | | | | | | |
| 321 | *Asp;Lys;Tyr;Val; | * is Conj- | 11 | 70 | — | 30 | 2266/763 | — | 5 |
| | Gly;NmLeu;Nle;NH2 | Pos1-C18DA | | | | | | | |
| 344 | *Asp;Lys;Tyr;Val; | * is Conj- | 3.1 | 95 | 260 | 60 | — | 10.3 | — |
| | Gly;NmLeu;Metox; | Neu-C18DA | | | | | | | |
| | NH2 | | | | | | | | |
| 390 | *Asp;Lys;Tyr;Val; | * is Conj- | 1 | 100 | 0.7 | 90 | — | 1.7 | — |
| | Gly;NmLeu;Nle;NH2 | Neu-C18MA | | | | | | | |
| 391 | *Asp;Lys;Tyr;Val; | * is Conj- | 0.9 | 100 | 0.6 | 90 | — | — | — |
| | Gly;NmLeu;Metox; | Neu-C18MA | | | | | | | |
| | NH2 | | | | | | | | |

Comparison of effect of protractor composition on NK2R-selective NKA(4-10) analogues on albumin binding in vitro, and compound exposure and pharmacokinetics in vivo to in vivo energy expenditure. The position of the protractors on the amino acid sequence is marked by an asterisk “*”, and the structures of the protractors/conjugated moieties can be found in Example 6.
BLLQ: below lower limit of quantification.
Conj: conjugate;
Neu: neutral charge;
Pos: positive charge;
Neg: negative charge;
MA: monoacid;
DA: diacid.
Cxx refers to the length of carbon atoms in lipid, i.e. C18 contains eighteen carbon atoms.

Results

Protractor Position
NKA(4-10) analogues, compounds 301 and 307, shows that Lys5 protracted analogues are active, but reduces NK2R potency compared to N-terminal protraction exemplified by compound 304.
Compound 302 with two protractors, on one peptide backbone in the N-terminal and at Lys5 inactivates the NKA(4-10) analogue (compound 302).

Linker Charge
The charge of the linker in the protractor between the fatty acid moiety and peptide backbone is important for receptor activation of the NKA(4-10) analogues.
Neutral charged linker does not modulate NK2R selectivity, potency and efficacy. (compounds: 304 and 305 versus 374 and 375).
Negative charged linker on N-terminal protractor or Lys5 decreases NK2R potency of the NKA(4-10) analogue (compounds: 319 and 333).
Positive charged linker, as in compounds 318 and 321, reduces potency and increases albumin binding causing lower exposure levels and decreased energy expenditure compared to neutral charged linker of compound 305.
Diacid fatty acid moiety on protractor is important for selectivity and half-life. Substituting from a C18 diacid, as in compounds 305 and 344, to a C18 monoacid fatty acid, as in compounds 390 and 391, completely destroys the NK2R selectivity and reduces half-life.
C18 diacid fatty acid length is optimal for NK2R potency and selectivity. Both reducing the C18 diacid fatty acid length on the protractor, as in compound 344, to C14 diacid (compound 368) or C16 diacid (compound 367), or prolonging to C20 diacid (compound 380), causes a decrease in NK2R selectivity and efficacy. Compounds 367 and 368 with C16 and C14 diacid respectively, also loses NK2R potency.

Summary

Based on the present example, it is concluded that a protractor constituting 2OEG-gammaGlu-C18 diacid positioned on N-terminal of NKA(4-10) analogues, as exemplified by compound 304, 305 and 344 is optimal for NK2R activation, selectivity, half-life and energy expenditure induction.

### Example 10 Investigation of NK2R-Selective NKA(4-10) Analogues on Weight Loss in Diet-Induced Obese Mice

Effect of protracted NKA analogues on energy expenditure and weight loss in diet-induced obese (DIO) mice.

Energy expenditure was measured in diet-induced obese mice housed in metabolic cages as described in example 8. Pharmacokinetics and exposure were measured in plasma from lean mice treated with compound as described in example 8.

TABLE 16

| | | In vivo efficacy | | | | |
|---|---|---|---|---|---|---|
| | | | | Energy | | Weight |
| | | | | expenditure | | loss |
| | | | | efficacy | | efficacy |
| | | | Energy | (change | | (change |
| | | Half- | expenditure | relative | Weight | relative |
| | | life | EC50 | to vehicle | loss EC50 | to vehicle |
| ID | Sequence | (h) | (nmol/kg) | control) | (nmol/kg) | control) |
| 304 | *Asp;Lys;Phe;Val; | 9.9 | 94 | 11% | 16 | 12% |
| | Gly;NmLeu;Nle;NH2 | | | | | |
| 305 | *Asp;Lys;Tyr;Val; | 5.5 | 34 | 11% | 69 | 6% |
| | Gly;NmLeu;Nle;NH2 | | | | | |
| 344 | *Asp;Lys;Tyr;Val; | 10.3 | 13 | 12% | 130 | 10% |
| | Gly;NmLeu;Metox; | | | | | |
| | NH2 | | | | | |
| 383 | *Asp;Lys;Phe;Thr; | 11.5 | 110 | 11% | 83 | 11% |
| | Gly;NmLeu;Metox; | | | | | |
| | NH2 | | | | | |

Comparison of half-life to in vivo potency and efficacy of NK2R-selective NKA(4-10) analogue 304 and highly NK2R-selective agonists, compounds 305, 344 and 383. The position of the protractor on the amino acid sequence is marked by an asterisk “*”, and the structures of the protractors/conjugated moieties can be found in Example 6.

Results
All NK2R-selective NKA(4-10) analogues (compound 304, 305, 344 and 383) increased energy expenditure with similar efficacy of 11-12% compared to vehicle.
Energy expenditure potency was improved in analogues with Tyr5 mutation (compound 305 and 344) compared to compounds with Phe in position 5 (304 and 383).
Weight loss efficacy of selective NK2R agonists is determined by half-life. Thus, compound 305 with a half-life of 5.5 hours has approximately 50% reduced weight loss efficacy compared to compound 304, 344 and 383 that all have a half-life of approximately 10 hours or more.
Highly NK2R-selective analogues, such as 305, 344 and 383, exhibited slightly lower weight loss potency compared to compound 304. That is probably due to residual NK1R activation.

Summary

The present example demonstrates that highly NK2R selective and long-lived compounds of the present disclosure, such as 344 and 383, are preferred for weight loss induction.

### Example 11: Investigation of the Effect of NK2-Selective NKA(4-10) Analogue on Glucose Metabolism in Diet-Induced Obese Mice

Effect of protracted highly NK2R-selective NKA(4-10) analogues on glucose and insulin tolerance in insulin resistant and pre-diabetic diet-induced obese (DIO) mice.

Glucose and insulin tolerance tests were performed on diet-induced obese mice as described in example 8.

Results

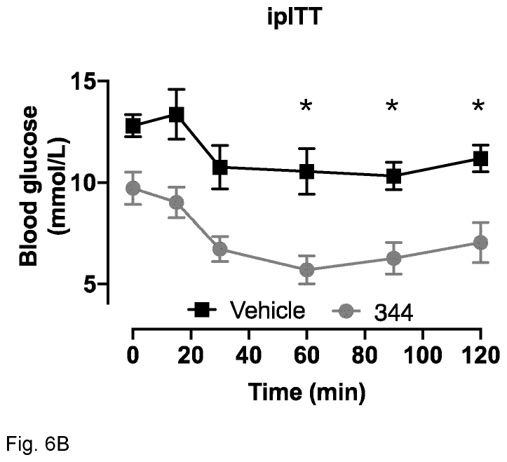

The results are shown in FIG. 6. The highly NK2R-selective molecule 344 improved both glucose and insulin tolerance in obese mice. The effect on insulin tolerance was driven by both a decrease in fasting blood glucose and increased insulin sensitivity.

Summary

Pharmacological NK2R activation improves glucose and insulin tolerance in diet-induced obese mice. The compounds of the present disclosure therefore have the potential to treat insulin resistance and diabetes.

### Example 12: Investigation of the Effect of NK2R-Selective NKA(4-10) Analogue on Dysfunctional Voiding in Wild-Type Mice

Effect of protracted highly NK2R-selective NKA(4-10) analogues on Loperamide-induced dysfunctional voiding in lean wild-type mice Loperamide-induced constipation was used as a model for dysfunctional voiding, as described in example 8.

Results

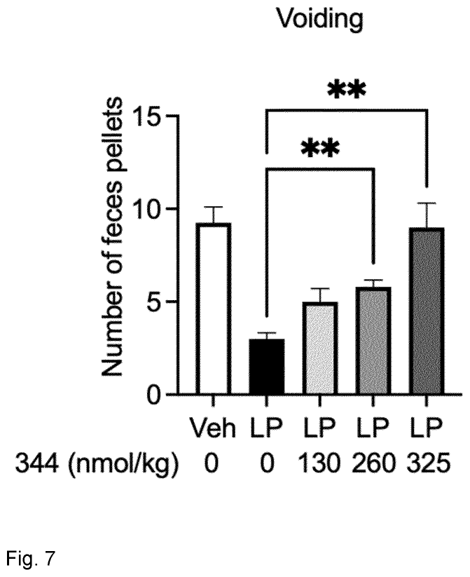

The results are shown in FIG. 7. The highly NK2R-selective molecule 344 improved Loperamide-induced dysfunctional voiding in a dose-dependent manner.

Summary

Pharmacological NK2R activation corrects dysfunctional voiding in mice. The compounds of the present disclosure therefore have the potential to treat dysfunctional voiding.

### Example 13: Sequences

| SEQ | | | |
|---|---|---|---|
| ID | | | |
| NO: | ID | Sequence | Notes |
| 1 | 301 | **Asp;Lys*;Phe;Val;Gly;NmLeu;Met;NH2 | **: acetyl; NmLeu: N- |
| | | | methyl-Leucine |
| 2 | 302 | *Asp;Lys*;Phe;Val;Gly;NmLeu;Met;NH2 | NmLeu: N-methyl-Leucine |
| 3 | 304 | *Asp;Lys;Phe;Val;Gly;NmLeu;Nle;NH2 | NmLeu: N-methyl- |
| | | | Leucine; Nle: norleucine |
| 4 | 305 | *Asp;Lys;Tyr;Val;Gly;NmLeu;Nle;NH2 | NmLeu: N-methyl- |
| | | | Leucine; Nle: norleucine |
| 5 | 306 | *Asp;Arg;Phe;Val;Gly;NmLeu;Nle;NH2 | NmLeu: N-methyl- |
| | | | Leucine; Nle: norleucine |
| 6 | 307 | Asp;Lys*;Phe;Val;Gly;NmLeu;Nle;NH2 | NmLeu: N-methyl- |
| | | | Leucine; Nle: norleucine |
| 7 | 308 | *Lys;Thr;Asp;Ser;Phe;Val;Gly;Leu;Nle;NH2 | Nle: norleucine |
| 8 | 310 | *Lys;Thr;Asp;Ser;Phe;Val;bAla;NmLeu;Nle;NH2 | bAla: beta alanine; |
| | | | NmLeu: N-methyl- |
| | | | Leucine; Nle: norleucine |
| 9 | 312 | *Lys;Thr;Asp;Ser;Phe;Val;Gly;NmLeu;Nle;NH2 | NmLeu: N-methyl- |
| | | | Leucine; Nle: norleucine |
| 10 | 313 | *Asp;Lys;Tyr;Val;Gly;NmLeu;cHexAla;NH2 | NmLeu: N-methyl- |
| | | | Leucine; cHexAla: L- |
| | | | cyclohexylalanine; |
| 11 | 314 | *Glu;Lys;Tyr;Arg;Gly;NmLeu;Nle;NH2 | NmLeu: N-methyl- |
| | | | Leucine; Nle: norleucine |
| 12 | 315 | *Glu;Lys;Phe;Arg;Gly;NmLeu;Nle;NH2 | NmLeu: N-methyl- |
| | | | Leucine; Nle: norleucine |
| 13 | 316 | *Asp;Lys;Tyr;Val;Gly;NmLeu;Met;NH2 | NmLeu: N-methyl-Leucine |
| 14 | 318 | *Asp;Lys;Tyr;Val;Gly;NmLeu;Nle;NH2 | *: Conj-Pos2; NmLeu: N- |
| | | | methyl-Leucine; Nle: |
| | | | norleucine |
| 15 | 319 | *Asp;Lys;Tyr;Val;Gly;NmLeu;Nle;NH2 | *: Conj-Neg; NmLeu: N- |
| | | | methyl-Leucine; Nle: |
| | | | norleucine |
| 16 | 321 | *Asp;Lys;Tyr;Val;Gly;NmLeu;Nle;NH2 | *: Conj-Pos1; NmLeu: N- |
| | | | methyl-Leucine; Nle: |
| | | | norleucine |
| 17 | 322 | *Lys;Thr;Asp;Ser;Phe;Val;bAla;Leu;Nle;NH2 | bAla: beta alanine; Nle: |
| | | | norleucine |
| 18 | 330 | *Asp;Lys;4-I-Phe;Val;Gly;NmLeu;Nle;NH2 | NmLeu: N-methyl- |
| | | | Leucine; Nle: norleucine |
| 19 | 334 | Asp;Lys*;Tyr;Val;Gly;NmLeu;Nle-NH2 | *: Conj-Neg; NmLeu: N- |
| | | | methyl-Leucine; Nle: |
| | | | norleucine |
| 20 | 335 | *Glu;Lys;Phe;Val;Gly;NmLeu;Nle;NH2 | NmLeu: N-methyl- |
| | | | Leucine; Nle: norleucine |
| 21 | 336 | *Glu;Lys;Tyr;Val;Gly;NmLeu;Nle;NH2 | NmLeu: N-methyl- |
| | | | Leucine; Nle: norleucine |
| 22 | 337 | *Glu;Arg;Phe;Val;Gly;NmLeu;Nle;NH2 | NmLeu: N-methyl- |
| | | | Leucine; Nle: norleucine |
| 23 | 344 | *Asp;Lys;Tyr;Val;Gly;NmLeu;Metox;NH2 | NmLeu: N-methyl- |
| | | | Leucine; Metox: |
| | | | metoxinine |
| 24 | 348 | *Asp;Lys;Tyr;Ile(S);Gly;NmLeu;Nle;NH2 | Ile(S): Ile S isoform; |
| | | | NmLeu: N-methyl- |
| | | | Leucine; Nle: norleucine |
| 25 | 351 | *Asp;Lys;Tyr;Ile(R);Gly;NmLeu;Nle;NH2 | Ile(R): Ile R isoform; |
| | | | NmLeu: N-methyl- |
| | | | Leucine; Nle: norleucine |
| 26 | 353 | *Asp;Lys;Tyr;Val;Gly;NmLeu;Phe;NH2 | NmLeu: N-methyl-Leucine |
| 27 | 356 | *Asp;Lys;dPhe;Val;Gly;NmLeu;Nle;NH2 | NmLeu: N-methyl- |
| | | | Leucine; Nle: norleucine |
| 28 | 357 | *Glu;Arg;Tyr;Val;Gly;NmLeu;Nle;NH2 | NmLeu: N-methyl- |
| | | | Leucine; Nle: norleucine |
| 29 | 361 | *Asp;Lys;3-OH-Phe;Val;Gly;NmLeu;Nle;NH2 | NmLeu: N-methyl- |
| | | | Leucine; Nle: norleucine |
| 30 | 362 | *Asp;Lys;Pro;Val;Gly;NmLeu;Nle;NH2 | NmLeu: N-methyl- |
| | | | Leucine; Nle: norleucine |
| 31 | 363 | *Asp;Lys;Val;Tyr;Gly;NmLeu;Nle;NH2 | NmLeu: N-methyl- |
| | | | Leucine; Nle: norleucine |
| 32 | 366 | *Asp;Lys;Tyr;Thr;Gly;NmLeu;Metox;NH2 | NmLeu: N-methyl- |
| | | | Leucine; Metox: |
| | | | metoxinine |
| 33 | 367 | *Asp;Lys;Tyr;Val;Gly;NmLeu;Nle;NH2 | *: Conj-Neu-C16; NmLeu: |
| | | | N-methyl-Leucine; Nle: |
| | | | norleucine |
| 34 | 368 | *Asp;Lys;Tyr;Val;Gly;NmLeu;Nle;NH2 | *: Conj-Neu-C14; NmLeu: |
| | | | N-methyl-Leucine; Nle: |
| | | | norleucine |
| 35 | 369 | *Asp;Lys;Tyr;Val;Gly;NmLeu;4-F-Phe;NH2 | NmLeu: N-methyl-Leucine |
| 36 | 370 | *Asp;Lys;Phe;Val;Gly;NmLeu;4-MeOPhe;NH2 | NmLeu: N-methyl- |
| | | | Leucine; 4-MeOPhe: L-4- |
| | | | Methoxyphenylalanine |
| 37 | 373 | *Asp;Lys;Phe;Val;Aib;Leu;Nle;NH2 | Aib: 2-aminoisobutyric |
| | | | acid |
| 38 | 374 | Asp;Lys;Tyr;Val;Gly;NmLeu;Nle;NH2 | NmLeu: N-methyl- |
| | | | Leucine; Nle: norleucine |
| 39 | 375 | Asp;Lys;Phe;Val;Gly;NmLeu;Nle;NH2 | NmLeu: N-methyl- |
| | | | Leucine; Nle: norleucine |
| 40 | 380 | *Asp;Lys;Tyr;Val;Gly;NmLeu;Nle;NH2 | *: Conj-Neu-C20; NmLeu: |
| | | | N-methyl-Leucine; Nle: |
| | | | norleucine |
| 41 | 381 | *Glu;Lys;Tyr;Thr;Gly;NmLeu;Metox;NH2 | NmLeu: N-methyl- |
| | | | Leucine; Metox: |
| | | | metoxinine |
| 42 | 382 | *Glu;Lys;Tyr;Val;Gly;NmLeu;Metox;NH2 | NmLeu: N-methyl- |
| | | | Leucine; Metox: |
| | | | metoxinine |
| 43 | 383 | *Asp;Lys;Phe;Thr;Gly;NmLeu;Metox;NH2 | NmLeu: N-methyl- |
| | | | Leucine; Metox: |
| | | | metoxinine |
| 44 | 384 | *Asp;Lys;Phe;Val;Gly;NmLeu;Metox;NH2 | NmLeu: N-methyl- |
| | | | Leucine; Metox: |
| | | | metoxinine |
| 45 | 385 | *Asp;Lys;Phe;Val;dSer;Leu;Nle;NH2 | Nle: norleucine |
| 46 | 386 | *Asp;Lys;Tyr;Ser;Gly;NmLeu;Nle;NH2 | NmLeu: N-methyl- |
| | | | Leucine; Nle: norleucine |
| 47 | 387 | *Asp;Lys;Phe;Thr;Gly;NmLeu;Nle;NH2 | NmLeu: N-methyl- |
| | | | Leucine; Nle: norleucine |
| 48 | 389 | *Asp;Lys;Phe;Val;Gly;Leu;Nle;NH2 | Nle: norleucine |
| 49 | 390 | *Asp;Lys;Tyr;Val;Gly;NmLeu;Nle;NH2 | NmLeu: N-methyl- |
| | | | Leucine; Nle: norleucine |
| 50 | 391 | *Asp;Lys;Tyr;Val;Gly;NmLeu;Metox;NH2 | NmLeu: N-methyl- |
| | | | Leucine; Metox: |
| | | | metoxinine |
| 51 | 392 | *Asp;Lys;Tyr;Val;Gly;Leu;Nle;NH2 | Nle: norleucine |
| 52 | 393 | *Asp;Lys;Tyr;Val;Gly;Leu;Metox;NH2 | Metox: metoxinine |
| 53 | 394 | *Asp;Lys;Tyr;Val;Aib;Leu;Metox;NH2 | Aib: 2-aminoisobutyric |
| | | | acid |
| 54 | 395 | *Asp;Lys;Tyr;Val;Aib;Leu;Nle;NH2 | Aib: 2-aminoisobutyric |
| | | | acid |
| 55 | 396 | *Asp;Lys;Tyr;Val;dSer;Leu;Metox;NH2 | NmLeu: N-methyl- |
| | | | Leucine; Metox: |
| | | | metoxinine |
| 56 | 397 | *Glu;Lys;Phe;Thr;Gly;NmLeu;Nle;NH2 | NmLeu: N-methyl- |
| | | | Leucine; Nle: norleucine |
| 57 | 402 | Asp;Lys;Tyr;Val;dSer;Leu;Nle;NH2 | Nle: norleucine |

Unless stated otherwise, “*” is “Conj-Neu-C18DA” the structure of which is illustrated in Example 6. The conjugated moiety is attached to the N-terminal α-amino group, unless stated otherwise.

1. A neurokinin receptor 2 (NK2R) agonist according to formula (I):
(A)-(B) (I),
wherein;
(A) is a peptide comprising an amino acid sequence of the general formula X₁X₂X₃X₄X₅X₆X₇, wherein
X₁ is selected from the group consisting of: aspartic acid (D) and glutamic acid (E);
X₂ is selected from the group consisting of: lysine (K), arginine (R), and histidine (H);
X₃ is selected from the group consisting of: tyrosine (Y), phenylalanine (F), meta-tyrosine (m-Y), valine (V), tryptophan (W), methionine (M), leucine (L), isoleucine (I), and alanine (A);
X₄ is selected from the group consisting of: valine (V), threonine (T), serine (S), asparagine (N), glutamine (0), glycine (G), and alanine (A);
X₅ is selected from the group consisting of: glycine (G), 2-aminoisobutyric acid (Aib), serine (S), alanine (A), valine (V), leuicine (L), beta-alanine (bA) and isoleucine (I);
X₆ is selected from the group consisting of: leucine (L), isoleucine (I), alanine (A) and N-methyl leucine (Me-Leu); and
X₇ is selected from the group consisting of: norleucine (Nle), methoxinine (Mox), methionine (M), 4-fluorophenylalanine (4fF), and 4-methoxyphenylalanine (4MeOF);
(B) is a conjugated moiety of the general formula (II)
Fa-Lg (II),
wherein;
Fa is of formula (Fa-1), 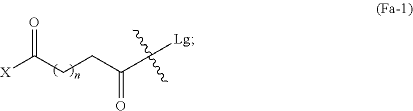
wherein n is from 14 to 17, preferably wherein n is 15;
and wherein X is selected from the group consisting of —OH, —OC₁₋₆, —NH₂, —NHC₁₋₆, and N(C₁₋₆)₂,
Lg is a linking group of formula (Lg-1), 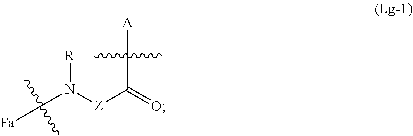
wherein Z is a chain comprising from 18 to 23 atoms in the backbone selected from the group consisting of: C, O, and N;
and wherein R is selected from the group consisting of H, and C₁₋₆ alkyl; and Lg covalently links (B) to the peptide (A),
and wherein (B) is covalently linked to a terminal amino acid.

2. The NK2R agonist according to any one of the preceding claims, wherein the peptide (A) is of the general formula X₁X₂X₃X₄X₅X₆X₇, wherein
X₁ is selected from the group consisting of: aspartic acid (D) and glutamic acid (E);
X₂ is selected from the group consisting of: lysine (K), and arginine (R);
X₃ is selected from the group consisting of: tyrosine (Y), and phenylalanine (F), and meta-tyrosine (m-Y),
X₄ is selected from the group consisting of: valine (V), and threonine (T);
X₅ is selected from the group consisting of: glycine (G), 2-aminoisobutyric acid (Aib), beta-alanine (bA) and serine (S);
X₆ is selected from the group consisting of: leucine (L), and N-methyl leucine (Me-Leu); and
X₇ is selected from the group consisting of: norleucine (Nle), methoxinine (Mox), methionine (M), 4-fluorophenylalanine (4fF), and 4-methoxyphenylalanine (4MeOF).

3. The NK2R agonist according to any one of the preceding claims, wherein X₂ is arginine (R).

4. The NK2R agonist according to any one of the preceding claims, wherein X₃ is tyrosine (Y).

5. The NK2R agonist according to any one of the preceding claims, wherein X₄ is threonine (T).

6. The NK2R agonist according to any one of the preceding claims, wherein X₅ is selected from the group consisting of: 2-aminoisobutyric acid (Aib) and serine (S).

7. The NK2R agonist according to any one of the preceding claims, wherein X₆ is N-methyl-leucine (Me-Leu).

8. The NK2R agonist according to any one of the preceding claims, wherein X₇ is methoxinine (Mox).

9. The NK2R agonist according to any one of the preceding claims, wherein n is 15 and wherein X is —OH.

10. The NK2R agonist according to any one of the preceding claims, wherein Lg of the conjugated moiety does not comprise functional groups that are positively charged at pH=7.4.

11. The NK2R agonist according to any one of the preceding claims, wherein Lg of the conjugated moiety has a net neutral charge or −1 at pH=7.4.

12. The NK2R agonist according to any one of the preceding claims, wherein the conjugated moiety is of formula (B1);

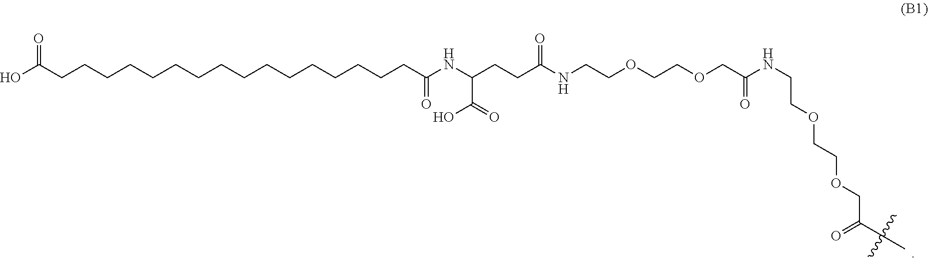 [SMILES: *C(=O)COCCOCCNC(=O)COCCOCCNC(=O)CCC(NC(=O)CCCCCCCCCCCCCCCCC(=O)O)C(=O)O]

13. The NK2R agonist according to any one of the preceding claims, wherein the conjugated moiety (B) is covalently attached to the N-terminus of (A), optionally via an amide bond.

14. The NK2R agonist according to any one of the preceding claims, wherein the conjugated moiety (B) is covalently attached to the N-terminus of (A) via an amide bond with the N-terminal α-NH₂ group.

15. The NK2R agonist according to any one of the preceding claims, wherein the peptide (A) is amidated on the C-terminus.

16. The NK2R agonist according to any one of the preceding claims, wherein the peptide (A) comprises from 7 to 15 amino acids, such as from 7 to 14 amino acids, such as from 7 to 13 amino acids, such as from 7 to 12 amino acids, such as from 7 to 11 amino acids, such as from 7 to 11 amino acids, such as from 7 to 10 amino acids, such as from 7 to 9 amino acids, such as from 7 to 8 amino acids, preferably wherein the peptide comprises 7 amino acids.

17. The NK2R agonist according to any one of the preceding claims, wherein the peptide (A) comprises no more than 15 amino acids, such as no more than 14 amino acids, such as no more than 13 amino acids, such as no more than 12 amino acids, such as no more than 11 amino acids, such as no more than 10 amino acids, such as no more than 9 amino acids, such as no more than 8 amino acids, such as no more than 7 amino acids.

18. The NK2R agonist according to any one of the preceding claims, wherein the peptide (A) consists of 7 amino acids of the general formula X₁X₂X₃X₄X₅X₆X₇.

19. The NK2R agonist according to any one of the preceding claims, wherein
(A) is: Asp;Lys;Phe;Val;Gly;NmLeu;Nle;NH2 (compound 305), and
(B) is of formula (B1) covalently attached to the N-terminal aspartate of (A).

20. The NK2R agonist according to any one of claims 1-18, wherein
(A) is: Asp;Lys;Tyr;Val;Gly;NmLeu;Metox;NH2 (compound 344), and
(B) is of formula (B1) covalently attached to the N-terminal aspartate of (A).

21. The NK2R agonist according to any one of claims 1-18, wherein the NK2R agonist consists of the sequence of any one of SEQ ID NO: 1 to SEQ ID NO: 57.

22. The NK2R agonist according to any one of claims 1-18, wherein the NK2R agonist is:

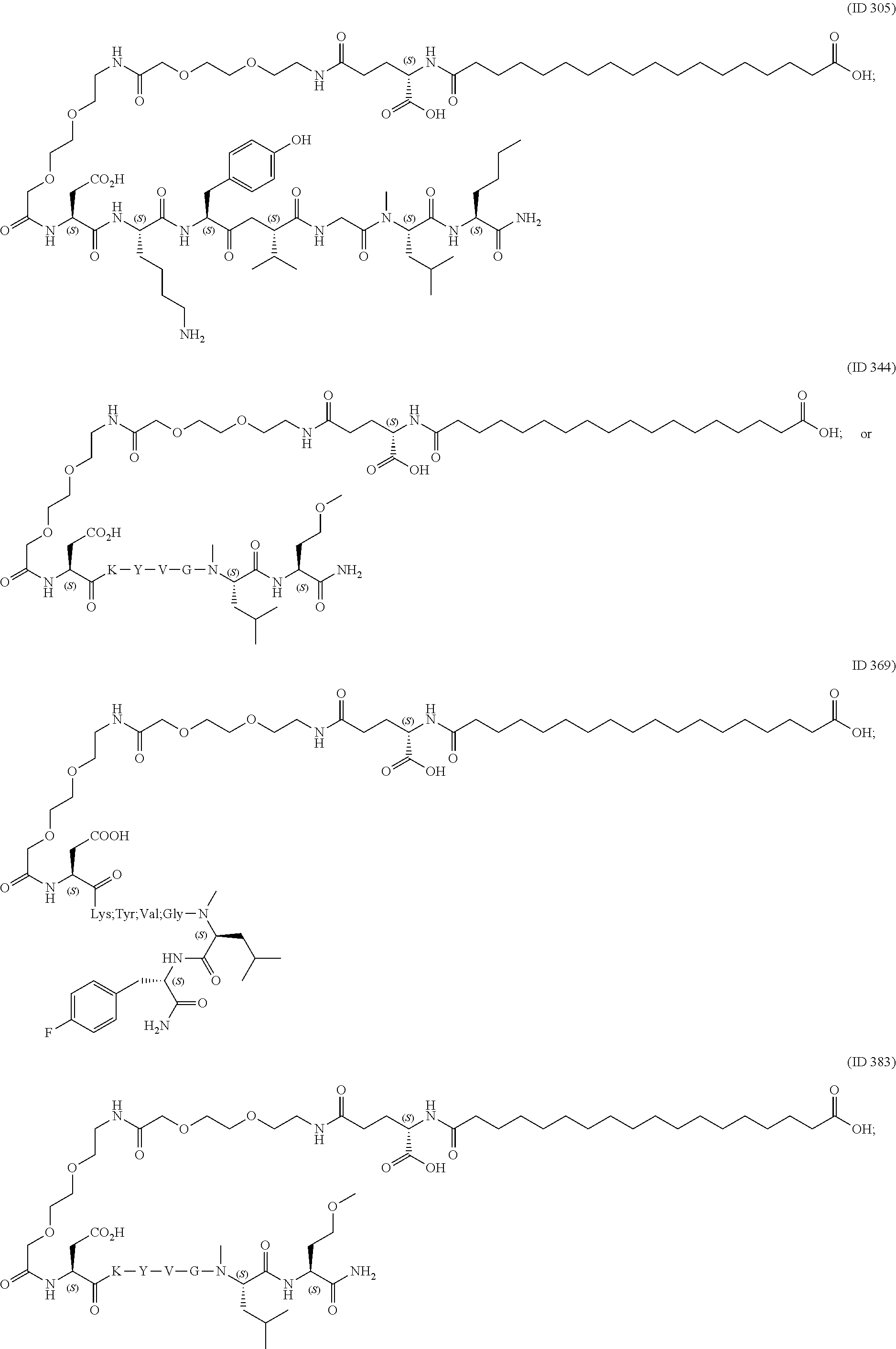

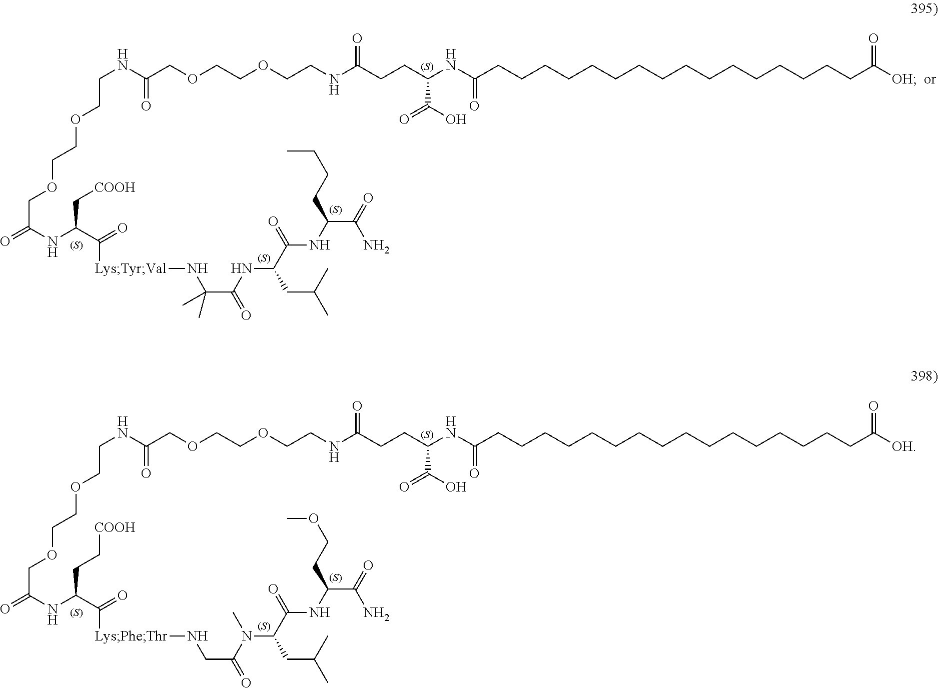

23. The NK2R agonist according to any one of the preceding claims, wherein the NK2R agonist is a selective neurokinin receptor 2 (NK2R) agonist.

24. The NK2R agonist according to any one of the preceding claims, wherein the NK2R agonist has an EC50 towards human NK2R of 300 nM or less, such as 250 nm or less, such as 200 nm or less, such as 150 nM or less, such as 100 nM or less, such as 90 nM or less, such as 80 nM or less, such as 70 nM or less, such as 60 nM or less, such as 50 nM or less.

25. The NK2R agonist according to any one of the preceding claims, wherein the NK2R agonist has an EC50 towards human NK2R of 50 nM or less, such as 40 nm or less, such as 30 nm or less, such as 20 nM or less, such as 15 nM or less, such as 14 nM or less, such as 13 nM or less, such as 12 nM or less, such as 11 nM or less, such as 10 nM or less.

26. The NK2R agonist according to any one of the preceding claims, wherein the NK2R agonist has an EC50 towards human NK1R of at least 100 nM, such as at least 200 nM, such as at least 300 nM, such as at least 400 nM, such as at least 500 nM.

27. The NK2R agonist according to any one of the preceding claims, wherein the NK2R agonist has an EC50 towards human NK3R of at least 100 nM, such as at least 200 nM, such as at least 300 nM, such as at least 400 nM, such as at least 500 nM.

28. A pharmaceutical composition comprising the neurokinin receptor 2 (NK2R) agonist as defined in any one of the preceding claims, and one or more pharmaceutically acceptable adjuvants, excipients, carriers, buffers and/or diluents.

29. A neurokinin receptor 2 (NK2R) agonist as defined in any one claims 1 to 27 for use as a medicament.

30. A method for treating a disease in a subject comprising administering a neurokinin receptor 2 (NK2R) agonist as defined in any one claims 1 to 29 for treatment of a NK2R mediated disorder.

31. The method according to claim 30, wherein the NK2R mediated disorder is selected from the group consisting of: obesity, dysfunctional voiding, diabetes, such as type-II diabetes, and diabetes-related disorders.

32. The method according to any one of claims 30-31, wherein the NK2R mediated disorder is a metabolic disorder.

33. The method according to any one of claims 30-32, wherein the metabolic disorder is a diabetes-related disorder.

34. The method according to claim 33, wherein the diabetes-related disorder is selected from the group consisting of: impaired insulin tolerance and impaired glucose tolerance.

35. A method for modulating the activity of NK2R, comprising contacting NK2R with a neurokinin receptor 2 (NK2R) agonist as defined in any one claims 1 to 27.

36. Use of a neurokinin receptor 2 (NK2R) agonist as defined in any one claims 1 to 27 for the manufacture of a medicament for the treatment of a metabolic disorder.


SEQUENCE LISTING (57 sequences)

SEQ ID NO: 1 - protein, 7 aa, Artificial
  Synthetic compound
  MOD_RES (1)..(1): ACETYLATION
  MISC_FEATURE (2)..(2): Conj-Neu-C18
  MOD_RES (6)..(6): N-methyl-Leucine
  MOD_RES (7)..(7): AMIDATION
DKFVGLM

SEQ ID NO: 2 - protein, 7 aa, Artificial
  Synthetic compound
  MISC_FEATURE (1)..(1): Conj-Neu-C18
  MISC_FEATURE (2)..(2): Conj-Neu-C18
  MOD_RES (6)..(6): N-methyl-Leucine
  MOD_RES (7)..(7): AMIDATION
DKFVGLM

SEQ ID NO: 3 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
DKFVGLL

SEQ ID NO: 4 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
DKYVGLL

SEQ ID NO: 5 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
DRFVGLL

SEQ ID NO: 6 - protein, 7 aa, Artificial
  Synthetic compound
  MISC_FEATURE (2)..(2): Conj-Neu-C18
  MOD_RES (6)..(6): N-methyl-Leucine
  MOD_RES (7)..(7): AMIDATION
  MOD_RES (7)..(7): Nle
DKFVGLL

SEQ ID NO: 7 - protein, 9 aa, Artificial
  Synthetic compound
  MISC_FEATURE (1)..(1): Conj-Neu-C18
  MOD_RES (7)..(7): AMIDATION
  MOD_RES (7)..(7): Nle
KTDSFVGLL

SEQ ID NO: 8 - protein, 9 aa, Artificial
  Synthetic compound
  MISC_FEATURE (1)..(1): Conj-Neu-C18
  MOD_RES (7)..(7): bAla
  MOD_RES (8)..(8): N-methyl-Leucine
  MOD_RES (9)..(9): Nle
  MOD_RES (9)..(9): AMIDATION
KTDSFVALL

SEQ ID NO: 9 - protein, 9 aa, Artificial
  Synthetic compound
  MISC_FEATURE (1)..(1): Conj-Neu-C18
  MOD_RES (8)..(8): N-methyl-Leucine
  MOD_RES (9)..(9): AMIDATION
  MOD_RES (9)..(9): Nle
KTDSFVGLL

SEQ ID NO: 10 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): L-cyclohexylalanine
DKYVGLA

SEQ ID NO: 11 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
EKYRGLL

SEQ ID NO: 12 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
EKFRGLL

SEQ ID NO: 13 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
DKYVGLM

SEQ ID NO: 14 - protein, 7 aa, Artificial
  Synthetic compound
  MISC_FEATURE (1)..(1): Conj-Pos2
  MOD_RES (6)..(6): N-methyl-Leucine
  MOD_RES (7)..(7): AMIDATION
  MOD_RES (7)..(7): Nle
DKYVGLL

SEQ ID NO: 15 - protein, 7 aa, Artificial
  Synthetic compound
  MISC_FEATURE (1)..(1): Conj-Neg
  MOD_RES (6)..(6): N-methyl-Leucine
  MOD_RES (7)..(7): AMIDATION
  MOD_RES (7)..(7): Nle
DKYVGLL

SEQ ID NO: 16 - protein, 7 aa, Artificial
  Synthetic compound
  MISC_FEATURE (1)..(1): Conj-Pos1
  MOD_RES (6)..(6): N-methyl-Leucine
  MOD_RES (7)..(7): AMIDATION
  MOD_RES (7)..(7): Nle
DKYVGLL

SEQ ID NO: 17 - protein, 9 aa, Artificial
  Synthetic compound
  MISC_FEATURE (1)..(1): Conj-Neu-C18
  MOD_RES (7)..(7): bAla
  MOD_RES (9)..(9): AMIDATION
  MOD_RES (9)..(9): Nle
KTDSFVALL

SEQ ID NO: 18 - protein, 7 aa, Artificial
  Synthetic compound
  MISC_FEATURE (1)..(1): Conj-Neu-C18
  MOD_RES (3)..(3): 4-I-Phe
  MOD_RES (6)..(6): N-methyl-Leucine
  MOD_RES (7)..(7): Nle
  MOD_RES (7)..(7): AMIDATION
DKFVGLL

SEQ ID NO: 19 - protein, 7 aa, Artificial
  Synthetic compound
  MISC_FEATURE (2)..(2): Conj-Neg
  MOD_RES (6)..(6): N-methyl-Leucine
  MOD_RES (7)..(7): AMIDATION
  MOD_RES (7)..(7): Nle
DKYVGLL

SEQ ID NO: 20 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
EKFVGLL

SEQ ID NO: 21 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
EKYVGLL

SEQ ID NO: 22 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
ERFVGLL

SEQ ID NO: 23 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MISC_FEATURE (7)..(6): Metoxinine
DKYVGLX

SEQ ID NO: 24 - protein, 7 aa, Artificial
  Synthetic compound
  MISC_FEATURE (1)..(1): Conj-Neu-C18
  MOD_RES (4)..(4): Ile S isoform
  MOD_RES (6)..(6): N-methyl-Leucine
  MOD_RES (7)..(7): AMIDATION
  MOD_RES (7)..(7): Nle
DKYIGLL

SEQ ID NO: 25 - protein, 7 aa, Artificial
  Synthetic compound
  MISC_FEATURE (1)..(1): Conj-Neu-C18
  MOD_RES (4)..(4): Ile R isoform
  MOD_RES (6)..(6): N-methyl-Leucine
  MOD_RES (7)..(7): AMIDATION
  MOD_RES (7)..(7): Nle
DKYIGLL

SEQ ID NO: 26 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
DKYVGLF

SEQ ID NO: 27 - protein, 7 aa, Artificial
  Synthetic compound
  MISC_FEATURE (1)..(1): Conj-Neu-C18
  MOD_RES (3)..(3): D-Phe
  MOD_RES (6)..(6): N-methyl-Leucine
  MOD_RES (7)..(7): AMIDATION
  MOD_RES (7)..(7): Nle
DKFVGLL

SEQ ID NO: 28 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
ERYVGLL

SEQ ID NO: 29 - protein, 7 aa, Artificial
  Synthetic compound
  MISC_FEATURE (1)..(1): Conj-Neu-C18
  MOD_RES (3)..(3): 3-OH-Phe
  MOD_RES (6)..(6): N-methyl-Leucine
  MOD_RES (7)..(7): AMIDATION
  MOD_RES (7)..(7): Nle
DKFVGLL

SEQ ID NO: 30 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
DKPVGLL

SEQ ID NO: 31 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
DKVYGLL

SEQ ID NO: 32 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Metoxinine
DKYTGLX

SEQ ID NO: 33 - protein, 7 aa, Artificial
  Synthetic compound
  MISC_FEATURE: Conj-Neu-C16
  MOD_RES (6)..(6): N-methyl-Leucine
  MOD_RES (7)..(7): AMIDATION
  MOD_RES (7)..(7): Nle
DKYVGLL

SEQ ID NO: 34 - protein, 7 aa, Artificial
  Synthetic compound
  MISC_FEATURE (1)..(1): Conj-Neu-C14
  MOD_RES (6)..(6): N-methyl-Leucine
  MOD_RES (7)..(7): AMIDATION
  MOD_RES (7)..(7): Nle
DKYVGLL

SEQ ID NO: 35 - protein, 7 aa, Artificial
  Synthetic compound
  MISC_FEATURE: Conj-Neu-C18
  MOD_RES (6)..(6): N-methyl-Leucine
  MOD_RES (7)..(7): AMIDATION
  MOD_RES (7)..(7): 4-F-Phe
DKYVGLF

SEQ ID NO: 36 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): L-4-Methoxyphenylalanine
DKFVGLF

SEQ ID NO: 37 - protein, 7 aa, Artificial
  Synthetic compound
  MISC_FEATURE (1)..(1): Conj-Neu-C18
  MISC_FEATURE (5)..(5): 2-aminoisobutyric acid
  MOD_RES (7)..(7): AMIDATION
  MOD_RES (7)..(7): Nle
DKFVXLL

SEQ ID NO: 38 - protein, 7 aa, Artificial
  Synthetic compound
  MOD_RES (6)..(6): N-methyl-Leucine
  MOD_RES (7)..(7): AMIDATION
  MOD_RES (7)..(7): Nle
DKYVGLL

SEQ ID NO: 39 - protein, 7 aa, Artificial
  Synthetic compound
  MOD_RES (6)..(6): N-methyl-Leucine
  MOD_RES (7)..(7): AMIDATION
  MOD_RES (7)..(7): Nle
DKFVGLL

SEQ ID NO: 40 - protein, 7 aa, Artificial
  Synthetic compound
  MISC_FEATURE (1)..(1): Conj-Neu-C20
  MOD_RES (6)..(6): N-methyl-Leucine
  MOD_RES (7)..(7): AMIDATION
  MOD_RES (7)..(7): Nle
DKYVGLL

SEQ ID NO: 41 - protein, 7 aa, Artificial
  Synthetic compound
  MISC_FEATURE (1)..(1): Conj-Neu-C18
  MOD_RES (6)..(6): N-methyl-Leucine
  MISC_FEATURE (7)..(7): Metoxinine
  MOD_RES (7)..(7): AMIDATION
EKYTGLX

SEQ ID NO: 42 - protein, 7 aa, Artificial
  Synthetic compound
  MISC_FEATURE (1)..(1): Conj-Neu-C18
  MOD_RES (6)..(6): N-methyl-Leucine
  MISC_FEATURE (7)..(7): Metoxinine
  MOD_RES (7)..(7): AMIDATION
EKYVGLX

SEQ ID NO: 43 - protein, 7 aa, Artificial
  Synthetic compound
  MISC_FEATURE (1)..(1): Conj-Neu-C18
  MOD_RES (6)..(6): N-methyl-Leucine
  MISC_FEATURE (7)..(7): Metoxinine
  MOD_RES (7)..(7): AMIDATION
DKFTGLX

SEQ ID NO: 44 - protein, 7 aa, Artificial
  Synthetic compound
  MISC_FEATURE (1)..(1): Conj-Neu-C18
  MOD_RES (6)..(6): N-methyl-Leucine
  MISC_FEATURE (7)..(7): Metoxinine
  MOD_RES (7)..(7): AMIDATION
DKFVGLX

SEQ ID NO: 45 - protein, 7 aa, Artificial
  Synthetic compound
  MISC_FEATURE (1)..(1): Conj-Neu-C18
  MOD_RES (5)..(5): D-Ser
  MOD_RES (7)..(7): AMIDATION
  MOD_RES (7)..(7): Nle
DKFVSLL

SEQ ID NO: 46 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
DKYSGLL

SEQ ID NO: 47 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
DKFTGLL

SEQ ID NO: 48 - protein, 7 aa, Artificial
  Synthetic compound
  MISC_FEATURE (1)..(1): Conj-Neu-C18
  MOD_RES (7)..(7): AMIDATION
  MOD_RES (7)..(7): Nle
DKFVGLL

SEQ ID NO: 49 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
DKYVGLL

SEQ ID NO: 50 - protein, 7 aa, Artificial
  Synthetic compound
  MISC_FEATURE (1)..(1): Conj-Neu-C18
  MOD_RES (6)..(6): N-methyl-Leucine
  MISC_FEATURE (7)..(7): Metoxinine
  MOD_RES (7)..(7): AMIDATION
DKYVGLX

SEQ ID NO: 51 - protein, 7 aa, Artificial
  Synthetic compound
  MISC_FEATURE (1)..(1): Conj-Neu-C18
  MOD_RES (7)..(7): AMIDATION
  MOD_RES (7)..(7): Nle
DKYVGLL

SEQ ID NO: 52 - protein, 7 aa, Artificial
  Synthetic compound
  MISC_FEATURE (1)..(1): Conj-Neu-C18
  MOD_RES (7)..(7): AMIDATION
  MISC_FEATURE (7)..(7): Metoxinine
DKYVGLX

SEQ ID NO: 53 - protein, 7 aa, Artificial
  Synthetic compound
  MISC_FEATURE (1)..(1): Conj-Neu-C18
  MISC_FEATURE (5)..(5): 2-aminoisobutyric acid
  MISC_FEATURE (7)..(7): Metoxinine
  MOD_RES (7)..(7): AMIDATION
DKYVXLX

SEQ ID NO: 54 - protein, 7 aa, Artificial
  Synthetic compound
  MISC_FEATURE (1)..(1): Conj-Neu-C18
  MISC_FEATURE (5)..(5): 2-aminoisobutyric acid
  MOD_RES (7)..(7): AMIDATION
  MOD_RES (7)..(7): Nle
DKYVXLL

SEQ ID NO: 55 - protein, 7 aa, Artificial
  Synthetic compound
  MISC_FEATURE (1)..(1): Conj-Neu-C18
  MOD_RES (5)..(5): D-Ser
  MISC_FEATURE (7)..(7): Metoxinine
  MOD_RES (7)..(7): AMIDATION
DKYVSLX

SEQ ID NO: 56 - protein, 7 aa, Artificial
  Synthetic compound
  MISC_FEATURE (1)..(1): Conj-Neu-C18
  MOD_RES (6)..(6): N-methyl-Leucine
  MOD_RES (7)..(7): AMIDATION
  MOD_RES (7)..(7): Nle
EKFTGLL

SEQ ID NO: 57 - protein, 7 aa, Artificial
  Synthetic compound
  MISC_FEATURE (1)..(1): Conj-Neu-C18
  MOD_RES (5)..(5): D-Ser
  MOD_RES (7)..(7): AMIDATION
  MOD_RES (7)..(7): Nle
DKYVSLL